(19) United States
(12) Patent Application Publication
(10) Pub. No.: US 2025/0249037 A1
(43) Pub. Date: Aug. 7, 2025

(54) CD70 BINDING CAR-T CELLS COMPRISING CD33 BINDING T-CELL ENGAGING ANTIBODY MOLECULES

(71) Applicant: The General Hospital Corporation, Boston, MA (US)

(72) Inventors: Marcela V. Maus, Lexington, MA (US); Mark Leick, Boston, MA (US)

(73) Assignee: The General Hospital Corporation, Boston, MA (US)

(21) Appl. No.: 18/856,583

(22) PCT Filed: Apr. 13, 2023
(86) PCT No.: PCT/US2023/065710

§ 371 Date: Oct. 11, 2024

Related U.S. Application Data
(60) Provisional application No. 63/331,758, filed on Apr. 15, 2022.
(60) Provisional application No. 63/341,995, filed on May 13, 2022.

(51) Int. Cl. A61K 35/17; A61K 40/11; A61K 40/31; A61K 40/33; A61K 40/42; A61P 35/02; C07K 14/705; C07K 14/725; C07K 16/28; C12N 15/85

(52) U.S. Cl. CPC A61K 35/17; A61K 40/11; A61K 40/31; A61K 40/33; A61K 40/421; A61K 40/4232; A61P 35/02; C07K 14/7051; C07K 14/70578; C07K 16/2803; C07K 16/2809; C07K 16/2875; C12N 15/85; A61K 2239/13; A61K 2239/21; A61K 2239/22; A61K 2239/48; C07K 2317/622; C07K 2319/02; C07K 2319/50; C12N 2800/22; C12N 2840/203

(57) ABSTRACT

The disclosure is directed to methods and compositions for treating cancers characterized by cells comprising chimeric antigen receptors (CARs) that bind CD70 and T-cell engaging antibody molecules (TEAMs) that bind CD33, nucleic acid molecules encoding chimeric antigen receptors (CARs) that bind CD70 and/or TEAMs that bind CD33, and compositions and methods related thereto.

### RELATED APPLICATIONS

This application claims the benefit under 35 U.S.C. § 119 (e) to U.S. Provisional Application No. 63/331,758, filed Apr. 15, 2022, and U.S. Provisional Application No. 63/341,995, filed May 13, 2022, the entire contents of each of which are incorporated herein by reference.

### GOVERNMENT FUNDING

This invention was made with government support under Grant No. 5R01CA238268-03, awarded by The National Institutes of Health. The government has certain rights in the invention.

### BACKGROUND

Chimeric Antigen Receptor T cells (CAR-T) have been highly effective for certain hematologic malignancies. The TNF-alpha family member CD70 has emerged as a promising surface target antigen in AML. However, antigen escape can occur after antigen-specific cancer treatment, leading to antigen-negative cancer relapse.

### SUMMARY

The disclosure is directed to methods and compositions for treating cancers characterized by cells expressing CD70 and optionally cells expressing a lower level of CD70 or that do not express CD70 (e.g., such that the cancer is no longer characterized by cells expressing CD70 in response to an anti-CD70 treatment (e.g., CD70 antigen escape)). The disclosure is directed, in part, to compositions and methods that overcome such antigen escape. The disclosure provides a dual targeting strategy in which a CD70 CAR T cell is engineered to express and secrete a T-cell engaging antibody molecule (TEAM). In some embodiments, the TEAM comprises a cancer binding moiety (e.g., anti-CD33 antibody) and an immune cell binding moiety (e.g., an anti-CD3 antibody). Without wishing to be bound by theory, the TEAM may direct binding of the CAR-T cell or bystander cells to the target cancer cells (e.g., cancer cells expressing CD33) or myeloid derived suppressor cells (MDSCs) (e.g., MDSCs expressing CD33) independent of CD70 CAR expression.

Accordingly, in one aspect, the disclosure is directed to a cell comprising a chimeric antigen receptor (CAR) that binds CD70 and a T-cell engaging antibody molecule (TEAM) that binds to CD33. In some embodiments, the cell is an immune cell. In some embodiments, the immune cell is a T-cell, a NK cell, a dendritic cell, a macrophage, a B cell, a neutrophil, an eosinophil, a basophil, a mast cell, a myeloid derived suppressor cell, a mesenchymal stem cell, a precursor thereof, or a combination. In some embodiments, the immune cell is a T-cell. In some embodiments, the cell is collected from a subject, optionally a human subject.

In some embodiments, the CAR comprises: (i) an extracellular target binding domain comprising a polypeptide that binds CD70; (ii) a transmembrane domain; and (iii) an intracellular signaling domain. In some embodiments, the extracellular target binding domain comprises the CD70-binding domain of CD27. In some embodiments, the extracellular target binding domain comprises the extracellular domain of CD27. In some embodiments, the extracellular target binding domain comprises an amino acid sequence that is at least 80% identical to the amino acid sequence of any one of SEQ ID NOs: 1, 8, or 9. In some embodiments, the extracellular target binding domain comprises the amino acid sequence of any one of SEQ ID NOs: 1, 8, or 9. In some embodiments, the extracellular target binding domain comprises an anti-CD70 antibody, optionally an scFv. In some embodiments, the transmembrane domain is the transmembrane domain of CD27. In some embodiments, the intracellular signaling domain comprises (i) an ITAM-containing signaling domains and/or (ii) one or more signaling domains from one or more co-stimulatory proteins or cytokine receptors. In some embodiments, the intracellular signaling domain comprises a CD3γ, CD3ε, CD3δ, or CD3ζ domain. In some embodiments, the intracellular signaling domain comprises a CD3ζ domain. In some embodiments, the costimulatory domain comprises a CD28, 4-1BB, 2B4, KIR, OX40, ICOS, MYD88, IL2 receptor, or SynNotch domain. In some embodiments, the costimulatory domain comprises a 4-1BB domain. In some embodiments, the extracellular target binding domain further comprises a signal peptide. In some embodiments the signal peptide comprises a CD27 signal peptide. In some embodiments, the CAR comprises an amino acid sequence that is at least 80% identical to the amino acid sequence of any one of SEQ ID NOs: 2-7. In some embodiments, the CAR comprises the amino acid sequence of any one of SEQ ID NOs: 2-7.

In some embodiments, the TEAM comprises an anti-CD33 antibody or a functional fragment thereof (e.g, a VH and/or VL domain of an anti-CD33 antibody). In some embodiments, the anti-CD33 antibody is selected from the group consisting of a fragment antigen-binding region (Fab region), a single-chain variable fragment (scFv), a diabody, a nanobody or a monoclonal antibody. In some embodiments, the anti-CD33 antibody is an scFv. In some embodiments, the anti-CD33 antibody comprises a VH domain having the amino acid sequence of SEQ ID NO: 20 and/or a VL domain having the amino acid sequence of SEQ ID NO: 19. In some embodiments, the VH domain is N-terminal of the VL domain. In some embodiments, the VL domain is N-terminal of the VH domain. In some embodiments, the TEAM comprises an immune cell binding moiety. In some embodiments, the immune cell binding moiety binds CD3, CD8, CD4, CXCR3, CCR4, GARP, LAP, CD25, CTLA-4, or CD16. In some embodiments, the immune cell binding moiety is selected from the group consisting of a fragment antigen-binding region (Fab region), a single-chain variable fragment (scFv), a diabody, a nanobody or a monoclonal antibody. In some embodiments, the immune cell binding moiety is an anti-CD3 scFv. In some embodiments, the TEAM comprises a linker between the anti-CD33 antibody and the immune cell binding moiety. In some embodiments, the linker is a non-cleavable linker, optionally a (GGGGS) 3 (SEQ ID NO: 27) linker. In some embodiments, the TEAM further comprises a secretion tag, optionally a IgK secretion tag. In some embodiments, the TEAM comprises an amino acid sequence that is at least 85% identical to any one of SEQ ID NOs: 17-18. In some embodiments, the cell comprises a polynucleotide molecule comprising a nucleic acid sequence encoding an amino acid sequence of any one of SEQ ID NOs: 17-18. In some embodiments, the nucleic acid sequence encoding the TEAM is codon-optimized.

In some embodiments, the cell comprises a first polynucleotide molecule comprising a nucleic acid sequence encoding the CAR and a second polynucleotide molecule comprising a nucleic acid sequence encoding the TEAM. In some embodiments, the cell comprises a polynucleotide molecule comprising a nucleic acid sequence encoding the CAR and a nucleic acid sequence encoding the TEAM. In some embodiments, the polynucleotide molecule further comprises a nucleic acid sequence encoding a linker between the nucleic acid sequence encoding the CAR and the nucleic acid sequence encoding the TEAM, optionally wherein the linker is a cleavable linker. In some embodiments, the cleavable linker is self-cleavable, optionally a P2A, E2A, F2A, or T2A self-cleavable linker. In some embodiments, the cleavable linker comprises a protease motif. In some embodiments, the linker comprises an internal ribosome entry site (IRES). In some embodiments, the polynucleotide molecule comprises a promoter operably linked to the nucleic acid sequence encoding the CAR and the nucleic acid sequence encoding the TEAM. In some embodiments, the promoter is a constitutively active promoter. In some embodiments, the promoter is an EF1alpha promoter.

In some embodiments, the sense strand of the polynucleotide molecule comprises, from 5′ to 3′, the nucleic acid sequence encoding the CAR, the linker, and the nucleic acid sequence encoding the TEAM. In some embodiments, the sense strand of the polynucleotide molecule comprises, from 5′ to 3′, the nucleic acid sequence encoding the TEAM, the linker, and the nucleic acid sequence encoding the CAR. In some embodiments, the polynucleotide molecule comprises a nucleic acid sequence encoding an amino acid sequence that is at least 85% identical to any one of SEQ ID NOs: 22-25.

In some aspects, the disclosure is directed to a polynucleotide comprising a nucleic acid sequence encoding the CAR and the TEAM as described herein. In some aspects, the disclosure is directed to a cell comprising the CAR and the TEAM as described herein.

In some aspects, the disclosure is directed to a method comprising administering to a subject the cell as described herein. In some aspects, the disclosure is directed to a method of treating a cancer characterized by cells expressing CD70, the method comprising administering to a subject in need thereof an effective amount of the cell as described herein.

In some aspects, the disclosure is directed to a method of treating a cancer characterized by cancer cells expressing CD70 and CD33, the method comprising administering to a subject in need thereof an effective amount of a cell as described herein.

In some aspects, the disclosure is directed to a method of treating a cancer characterized by cancer cells expressing CD33, the method comprising administering to a subject in need thereof an effective amount of a cell as described herein.

In some aspects, the disclosure is directed to a method of treating a cancer characterized by cancer cells that have decreased CD70 expression, the method comprising administering to a subject in need thereof an effective amount of a cell as described herein.

In some embodiments, the subject is human. In some embodiments, administering comprises infusion. In some embodiments, the cancer is a hematological cancer. In some embodiments, the cancer is a myeloid cancer. In some embodiments, the cancer is acute myeloid leukemia.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows a diagram of trCD27-CD8H/TM CAR secreting a CD33-CD3 bispecific T-cell engager (or T-cell engaging antibody molecule (TEAM)). The CD27 CAR can bind to acute myeloid leukemia (AML) and the CD33 TEAM can engage a bystander T Cell to the AML cell.

FIG. 2 shows graphs of flow cytometry data showing CD70 low relapse after CD70-targeted CAR treatment in an AML PDX. NSG mice were engrafted with 5×10⁶ DFAM68555 AML patient derived xenograft cells on day −4. On day 0 mice received 2×10⁶ trCD27 CD8H/TM modified CD70-targeted CAR-T cells. Most trCD8H/TM modified mice cleared tumor, however some mice relapsed with CD70 low tumor.

FIGS. 3A-3C show paired expression of CD33 and CD70 as combinatorial CAR targets in AML. FIG. 3A shows co-expression of CD33 and CD70 in blood. FIG. 8B shows expression of CD33 and CD70, alone and together, in primary AML samples. FIG. 3C shows the co-expression of the CD33+CD70 antigen pair in AML cells compared with normal BM HSCs and T cells.

FIG. 4 is a schematic demonstrating a putative advantage of a CARTEAM. CARTEAM cells demonstrate rapid renal clearance, spare hepatocytes and have decreased hepatoxicity.

FIGS. 5A-5E show diagrams of exemplary CAR and TEAMs and graphs demonstrating that TEAMs are secreted by CAR-T cells and bind to appropriate target antigens. FIG. 5A Structures of trCD27 CAR and/or CD33 TEAM constructs. FIG. 5B transduction efficiency of activated T-cells healthy donor T-cells (n=3 distinct healthy donors, and representative of at least 2 separate experiments). FIG. 5C supernatant from the indicated cells (rows) were added to the indicated cell types (column) and assessed for anti-His tag (TEAM) binding. FIG. 5D supernatant from Jurkat T cells transduced with the indicated constructs were incubated with plate bound Molm13 tumor cells as well as anti-His tag AF647 antibody (red). Fluorescent intensity was measured on an Incucyte with representative images as shown. FIG. 5E supernatant from Jurkat T cells transduced with the indicated constructs were co-cultured with Molm13 tumor cells and untransduced (UTD) healthy primary human T cells and anti-CD69 antibody. Percent of cells expressing CD69 across time are shown. PMA=phorbol 12-myristate 13-acetate positive control.

FIG. 6 shows diagrams of CAR-T cell and TEAM action and graphs demonstrating that CD33 TEAMs mediate UTD-driven, target-dependent cytotoxicity. Cells transduced with the trCD27 CAR-CD33-TEAM or trCD27-CD19-TEAM construct, or untransduced T-cells (UTD) were added to the top of a transwell insert. Untransduced T-cells were added with Molm13WT or Molm13CD33-cells to the bottom of the transwell insert. Only the trCD27-CAR-CD33-TEAM construct was able to mediate Molm13WT cell clearance, which was target dependent and was abrogated with loss of CD33.

FIGS. 7A-7B show graphs and micrographs demonstrating simultaneous T-cell redirection through CARs and TEAMs is efficacious against heterogeneous tumors and outperform a mix of individual CARs. FIG. 7A Individually targeted CD70 or CD33 CAR-T cells were compared to CD70CARCD33TEAM or CD70CARCD19TEAMs against AML targets with different levels of CD70 or CD33 (left). CD70CARCD33 TEAM cells exhibited superior cytotoxicity and CAR expansion in this real-time cytotoxicity assay on the incucyte. Representative images from the end of the experiment are shown in FIG. 7B. Tumor cells are green. Displayed data representative of CAR-T cells manufactured from 3 healthy donors' T-cells performed in duplicate. Experiment repeated at least n=2 times.

FIGS. 8A-8E show CD70CARCD33TEAM eradicates mixed tumor populations in vivo. FIG. 8A shows that NSG mice were injected with 5×10⁵ mixed population Molm13 cells (90% Molm13^{CD70KO}, 10% Molm13^{WT}, n=10 mice/group) on day-7 which express the bioluminescent (BLI) reporter click beetle green. On day 0 mice received 2×10⁶ of either CD70CAR^{CD19TEAM} or CD70CAR^{CD33TEAM} cells (n=5 mice per individual T-cell donor per group). FIGS. 8B-8C show serial BLI monitoring revealed tumor eradication in the CD70CAR^{CD33TEAM} group of mice, but uncontrolled tumor growth in the CD70CAR^{CD19TEAM} P value as indicated by 2-way ANOVA. FIG. 8D shows peripheral blood expansion determined by flow cytometry days 14-28 after CAR-T injection. P-values reported as the result of individual Mann-Whitney U tests. FIG. 8E shows peripheral blood CAR-T phenotypic profiling via flow cytometry from the blood of mice at day+21 after CAR-T injection compared to unstimulated and nontransduced, donor matched T-cells. Representative examples shown. CM (central memory), EM (effector memory), TDE (terminal differentiated effectors).

FIGS. 9A-9C shows CD70CAR^{CD33TEAM} cells exhibit divergent expressional programs compared to CD70CAR^{CD19TEAM} cells in vivo. FIG. 9A shows a volcano plot highlighting differences in transcriptional programs between the constructs. Genes with adjusted p-value less than 0.05 (Benjamini-Hochberg) are colored red. FIG. 9B shows pathway clustering was performed in an unbiased manner integrating all available pathways (top). Heatmap shown below for selected pathways. FIG. 9C shows pathway score for TCR-signaling is shown for CD70CAR^{CD33TEAM} (right) and CD70CAR^{CD19TEAM} (left). P-value by unpaired t-test.

FIG. 10 shows administration of two different doses of the CD70CAR^{CD33TEAM} CAR T cells to a patient derived xenograph (PDX) AML mouse model. 5×10⁶ AML PDX cells were administered to the mouse at day −4. At day zero, 5×10⁵ or 1×10⁶ CD70CAR^{CD33TEAM} CAR T cells were administered to the mice. Untreated and CD70CAR^{CD19TEAM} were used as controls. On day 15, tumor cell (CD33+/CD45+) concentration was measured in blood from the mice. Results showed that administering 5×10⁵ and 1×10⁶ CD70CAR^{CD33TEAM} CAR T cells had similar effects on tumor cell concentration.

FIGS. 11A-11E show construction of a CD70-CAR-T cell secreting a CD33 T cell engaging antibody molecule. FIG. 11A shows NSG mice were engrafted with 5×10⁶ DFAM68555 AML patient derived xenograft cells on day −4. On day 0 mice received 2×10⁶ CD70-targeted CAR-T cells. Most CAR-T cell treated mice cleared tumor, however some mice relapsed with CD70^{low} tumor. FIG. 11B shows CD70 CAR-T cell secreting a CD33-CD3 T cell engaging antibody molecule. FIG. 11D shows construct diagrams of CARs used herein. FIG. 11E shows transduction efficiency of primary healthy donor T-cells (n=3 distinct healthy donors, and representative of at least 2 separate experiments).

FIGS. 12A-12D show TEAMs are secreted by CAR-T cells and bind to appropriate target antigens. FIG. 12A shows supernatant from the indicated conditions (rows) were added to the indicated cell types (columns) and assessed for anti-His tag (TEAM) binding by flow cytometry. FIG. 12B shows supernatant from Jurkat T cells transduced with the indicated constructs were incubated with plate bound CD33-expressing K562 tumor cells as well as anti-His tag AF647 antibody. Fluorescent intensity was measured on an Incucyte with representative images as shown. FIG. 12C shows supernatant from Jurkat T cells transduced with the indicated constructs were co-cultured with Molm13 tumor cells, untransduced (UTD) healthy primary human T cells, and anti-CD69 antibody. Percent of cells expressing CD69 over time are shown. PMA=phorbol 12-myristate 13-acetate positive control. FIG. 12D shows cells transduced with the 70³³ or 70¹⁹ constructs, or UTD were added to the top of a transwell insert. UTD were added with Molm13^{WT} or Molm13^{CD33KO} cells to the bottom of the transwell insert. Tumor proliferation was measured over time. P-values represent 2 way ANOVA between the indicated conditions. Experiments repeated at least n=2 times.

FIGS. 13A-13C show simultaneous T-cell redirection through CARs and TEAMs is efficacious against heterogeneous tumors and outperform a mix of individual CARs. FIG. 13A shows individually targeted CAR33 T cells were compared to 70³³ or 70¹⁹ CAR-T cells against engineered Molm13 wt, Molm13^{CD70KO}, Molm13^{CD33KO} AML targets at a 2:1 effector:target ratio. Flow cytometric assessed expression of indicated targets is shown (left) and results of real time cytotoxicity assay (FIG. 13B-top) and CAR-T expansion (FIG. 13B-bottom). FIG. 13C shows representative images from the incucyte at 40 hours for the indicated conditions. Displayed data represent combined data from CAR-T cells manufactured from 3 healthy donors' T-cells performed in duplicate. Experiment repeated at least n=2 times. P-values represent 2-way ANOVA of the indicated conditions.

FIG. 14A-14D shows simultaneous T-cell redirection through CARs and TEAMs outperforms standard CAR-T cells in a patient derived xenograft model of AML. FIG. 14A shows 5×10⁶ DFAM68555 AML PDX cells were injected by tail vein on day-4 into NSG mice (n=5 mice/group). Four days later on day 0 5×10⁵ or 1×10⁶ 70³³ or 70¹⁹ CAR-T cells were injected by tail vein. FIG. 14B shows circulating CD33+ tumor cells are killed with higher efficacy by the CD70 CAR-T cell secreting a CD33 TEAM (70³³) than the CD70 CAR-T cell secreting a CD19 TEAM (70¹⁹). FIG. 14C shows the number of circulating 70³³ and 70¹⁹ CAR-T cells at day +14. FIG. 14D Survival of mice in the experiment. P-value represents log-rank mantel cox.

FIGS. 15A-15H show that 70³³ eradicates mixed tumor populations in vivo. FIG. 15A shows NSG mice were injected with 5×10⁵ mixed population Molm13 cells (90% Molm13^{CD70KO}, 10% Molm13^{WT}, n=10 mice/group) on day −7 which express the bioluminescent (BLI) reporter click beetle green. On day 0 mice received 2×10⁶ of either 70¹⁹ or 70CAR³³ cells (n=5 mice per individual T-cell donor per group). FIG. 15B shows peripheral blood expansion of CAR-T cells determined by flow cytometry days 14-28 after CAR-T injection. FIGS. 15C-15D show serial BLI monitoring of tumor growth. P-value represents 2-way ANOVA. FIG. 15E show peripheral blood CAR-T phenotypic profiling via flow cytometry from the blood of mice at day +21 after CAR-T injection compared to unstimulated and nontransduced, donor matched T-cells. Representative examples shown. CM (central memory), EM (effector memory), TDE (terminal differentiated effectors). FIG. 15F shows FACS sorted CAR-T cells from the spleens of the mice at day +28 from the mixed tumor model underwent gene expression analysis via nanostring with the CAR-T characterization panel in addition to a custom ‘drop-in’ gene set. Volcano plot highlighting differences in transcriptional programs between the constructs. Genes with adjusted p-value less than 0.05 (Benjamini-Hochberg) are colored dark grey. FIG. 15G shows pathway hierarchical clustering as performed in an unbiased manner integrating all available pathways (top). Heatmap shown for selected pathways. FIG. 15H shows pathway score for TCR-signaling is shown for 70^{33T} and 70CAR¹⁹. P-value by unpaired t-test.

FIGS. 16A-16G show TEAMs effectively redirect AML patients' T cells to kill tumor cells. FIG. 16A shows clinical characteristics of AML patients whose T cells were utilized. FIG. 16B shows flow T cell phenotyping of AML patient T-cells relative to healthy donor T cells. FIG. 16C shows representative phenotyping and PD-1 expression among AML patients' T cells. FIG. 16D shows co-culture of TEAM producer cells with isolated AML patients' T cells and Molm13 tumor targets. Real time cytotoxicity measured on the incucyte. FIG. 16E shows normalized cytokine measurement after 18 hours obtained from patients relative to healthy donor. FIG. 16F shows transwell assay with TEAM producer cells on top and isolated AML patient T cells on bottom with tumor targets. Real time cyototoxicity is measured (top) and activation (CD69) bottom. FIG. 16G shows representative images at 40 hours of activation.

FIG. 17 shows simultaneous T-cell redirection through CARs and TEAMs is efficacious against heterogeneous tumors with endogenous antigen expression levels and outperform a mix of individual CARs. Individually targeted CAR33 T cells were compared to 70³³ or 70¹⁹ CAR-T cells against the AML cell lines Kasumi1, Monomac1, and OCI-AML3 at a 2:1 effector:target ratio. Flow cytometric assessed expression of indicated targets is shown (left) and results of real time cytotoxicity assay (top) and CAR-T expansion (bottom). Displayed data represent combined data from CAR-T cells manufactured from 3 healthy donors' T-cells performed in duplicate. Experiment repeated at least n=2 times. P-values represent 2-way ANOVA of the indicated conditions.

FIG. 18 shows pathway hierarchical clustering was performed in an unbiased manner integrating all available pathways (top). Heatmap showing all nanostring available pathways.

### DETAILED DESCRIPTION

### CD-70 Binding Chimeric Antigen Receptors (CARs)

The present disclosure, in some aspects, provides CAR-T cells targeting the tumor necrosis alpha family member, CD70, and the use of the CAR-T cells for the treatment of hematologic malignancies (e.g., acute myeloid leukemia (AML)). CD70 is consistently expressed on myeloid blasts and leukemic stem cells but is highly restricted expression in healthy human tissues. As demonstrated previously, CD70-targeting CAR-T cells achieved antigen-specific activation, cytokine production, and cytotoxic activity in models of leukemia in vitro and in vivo, e.g., as described in PCT/US2020/051018, which is incorporated by reference in its entirety.

Some aspects of the present disclosure provide chimeric antigen receptors (CARs) comprising: (i) an extracellular target binding domain comprising a polypeptide that binds CD70; (ii) a transmembrane domain; and (iii) an intracellular signaling domain.

A “chimeric antigen receptor (CAR)” refers to a receptor protein that has been engineered to perform both antigen-binding and cell activating functions. In some embodiments, a CAR comprises a plurality of linked domains having distinct functions. CAR domains include those with antigen-binding functions, those with structural functions, and those with signaling functions. In some embodiments, a CAR comprises at least an extracellular ligand domain, a transmembrane domain and a cytoplasmic signaling domain (also referred to herein as “an intracellular signaling domain”) comprising a functional signaling domain derived from a stimulatory molecule as defined below. In some embodiments, the CAR comprises an optional leader sequence (also referred to as “signal peptide”), an extracellular antigen binding domain, a hinge, a transmembrane domain, and an intracellular stimulatory domain. In some embodiments, the domains in the CAR are in the same polypeptide chain, e.g., comprise a chimeric fusion protein. In some embodiments, the domains in the CAR are not contiguous with each other.

In some embodiments, the CAR described herein comprises an extracellular target binding domain comprising a polypeptide that binds Cluster of Differentiation 70 (CD70). “CD70” refers to a polypeptide that is encoded by the human CD70 gene (NCBI Gene ID: 970). As described herein, expression of CD70 is highly restricted in normal human (non-cancer) tissues. However, CD70 is expressed in numerous cancers, for example, bladder cancer, breast invasive carcinoma, cervical cancer, cholangiocarcinoma, colorectal cancer, diffuse large B-cell lymphoma (DLBC), Esophagus, glioblastoma (GBM), head and neck cancer, low-grade gliomas (LGG), liver cancer, lung adeno cancer, melanoma, mesothelioma, ovarian cancer, pancreatic cancer, prostate cancer, sarcoma, stomach cancer, testicular germ cell cancer, thymoma, thyroid cancer, uterine cancer, uveal melanoma, clear cell renal cell carcinoma (ccRCC), chromophobe renal cell carcinoma, papillary renal cell carcinoma (pRCC), acute myeloid leukemia, and adenoid cystic carcinoma (ACC) (Pan-Cancer Atlas 2018). CD70 is a cytokine that contains a cytoplasmic, transmembrane, and extracellular domains. The extracellular domain of CD70 is a ligand for CD27.

In some embodiments, the polypeptide that binds CD70 comprises a CD70-binding domain of Cluster of Differentiation 27 (CD27) also called the CD27 antigen. “CD27” refers to a polypeptide that is encoded by the human CD27 gene (NCBI GENE ID: 939, Uniprot ID: P26842). An example of the CD27 amino acid sequence is provided below.

(SEQ ID NO: 8)
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGMF
LVFTLAGALFLHQRRKYRSNKGESPVEPAEPCHYSCPREEEGSTIPIQED
YRKPEPACSP

The CD27 protein has extracellular, transmembrane, and cytoplasmic domains. In some embodiments, the CD70 binding domain is located within the extracellular signaling domain of CD27. In some embodiments, the extracellular region contains multiple cysteine-rich domains (CRD): CDR1, CDR2, and CDR3. In some embodiments, the CD70 binding domain is located within the CRD2 domain.

In some embodiments, the CD70-binding domain in CD27 comprises a peptide comprising the amino acid sequence of TRPHCESCRHCN (SEQ ID NO: 9) that is located in the extracellular domain of CD27. In some embodiments, the extracellular targeting binding domain of the CAR described herein comprises a polypeptide comprising an amino acid sequence that is at least 70% identical (e.g., at least 70%, at least 75%, at least 80%, at least 85%, at least 90%, at least 95% or at least 99% identical) to the amino acid sequence of SEQ ID NO: 9. In some embodiments, the extracellular targeting binding domain of the CAR described herein comprises the amino acid sequence of SEQ ID NO: 9.

In some embodiments, the extracellular targeting binding domain of the CAR described herein comprises a polypeptide comprising the extracellular domain of CD27 or a functional fragment thereof (e.g., a fragment capable of binding CD70). In some embodiments, a functional fragment comprises SEQ ID NO: 9, or a variant thereof. In some embodiments, the extracellular targeting binding domain of the CAR described herein comprises a polypeptide comprising an amino acid sequence that is at least 70% identical (e.g., at least 70%, at least 75%, at least 80%, at least 85%, at least 90%, at least 95% or at least 99% identical) to the amino acid sequence of SEQ ID NO: 1, 8 or 9. In some embodiments, the extracellular targeting binding domain of the CAR described herein comprises a polypeptide comprising the amino acid sequence of SEQ ID NO: 1.

In some embodiments, the polypeptide that binds CD70 in the extracellular targeting binding domain of the CAR described herein comprises an anti-CD70 antibody. The term “antibody,” used herein encompasses antibodies of different formats and antibody fragments. In some embodiments, antibody includes but is not limited to a monoclonal antibody, a polyclonal antibody, a recombinant antibody, a human antibody, a humanized antibody, and a functional fragment thereof, including but not limited to a single-chain variable fragment (scFV), a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the polypeptide that binds CD70 in the extracellular targeting binding domain of the CAR described herein comprises a scFv that binds to CD70.

In some embodiments, the CD70 antibody comprises an antibody or antigen binding domain (e.g., CDRs or VH and VL) as described in U.S. Pat. Nos. 11,434,298, 7,491,390, 8,124,738, 11,377,500, or U.S. Pat. No. 9,701,752, each of which is incorporated by reference in its entirety.

In some embodiments, the antibody is a human antibody or an antibody fragment. In some embodiments, the antibody a humanized antibody or an antibody fragment. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28 (4/5): 489-498; Studnicka et al., 1994, Protein Engineering, 7 (6): 805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is non-human. These non-human amino acid residues are often referred to as “import” residues, which are typically taken from an “import” variable domain. As provided herein, humanized antibodies or antibody fragments comprise one or more CDRs from non-human immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a non-human species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592, 106; EP 519,596; Padlan, 1991, Molecular Immunology, 28 (4/5): 489-498; Studnicka et al., Protein Engineering, 7 (6): 805-814 (1994); and Roguska et al., PNAS, 91:969-973 (1994)) or chain shuffling (U.S. Pat. No. 5,565,332), the contents of which are incorporated herein by reference herein in their entirety.

In some embodiments, the antibody is derived from a display library. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g., from three amino acids to over 300 amino acids. A display library entity can include more than one polypeptide component, for example, the two polypeptide chains of a Fab. In one exemplary embodiment, a display library can be used to identify an antigen binding domain. In a selection, the polypeptide component of each member of the library is probed with the antigen, or a fragment there, and if the polypeptide component binds to the antigen, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the phage display. In phage display, the protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; WO 92/18619; WO 91/17271; WO 92/20791; WO 92/15679; WO 93/01288; WO 92/01047; WO 92/09690; WO 90/02809. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced. Other display formats include cell based display (see, e.g., WO 03/029456), protein-nucleic acid fusions (see, e.g., U.S. Pat. No. 6,207,446), ribosome display, and E. coli periplasmic display.

The transmembrane domain of the CARs described herein may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD27, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, rfGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, and CD19. In some embodiments, the transmembrane domain is a CD28 transmembrane domain or CD8 transmembrane domain. In some embodiments, transmembrane domain is the transmembrane domain of CD27. In some embodiments, the transmembrane domain of CD27 comprises an amino acid sequence of ILVIFSGMFLVFTLAGALFL (SEQ ID NO: 10).

In some embodiments, the transmembrane domain can be attached to the extracellular region of the CAR, e.g., the ligand domain of the CAR, via a hinge, e.g., a hinge from a human protein. For example, in one embodiment, the hinge can be a human Ig (immunoglobulin) hinge, e.g., an IgG4 hinge, or a CD8a hinge.

In some embodiments, the cytoplasmic domain or region of the CAR described herein includes one or more intracellular signaling domains. An intracellular signaling domain is capable of activation of at least one of the normal effector functions of the immune cell in which the CAR has been introduced. Examples of intracellular signaling domains for use in the CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

An “intracellular signaling domain,” as the term is used herein, refers to an intracellular portion of a molecule. The intracellular signaling domain can generate a signal that promotes an immune effector function of the CAR containing cell, e.g., a CAR T cell or CAR-expressing NK cell. Examples of immune effector function, e.g., in a CAR T cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the one or more intracellular signaling domains comprise a primary intracellular signaling domain. Exemplary primary intracellular signaling domains include those derived from the molecules responsible for primary stimulation, or antigen dependent simulation. In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD3 theta, CD3 eta, CD5, CD22, CD79a, CD79b, CD278 (“ICOS”), FceRI, CD66d, DAP10, and DAP12. In some embodiments, the intracellular signaling domain of the CAR comprises a CD3-zeta (CD3ζ) signaling domain. In some embodiments, the CD3-zeta (CD3ζ) signaling domain comprises the amino acid sequence of: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALP PR (SEQ ID NO: 11). In some embodiments, the CD3-zeta (CD3ζ) signaling domain of the CAR described herein comprises an amino acid sequence that is at least 70% identical (e.g., at least 70%, at least 75%, at least 80%, at least 85%, at least 90%, at least 95% or at least 99% identical) to the amino acid sequence of SEQ ID NO: 11.

In some embodiments, the one or more intracellular signaling domain comprise a costimulatory intracellular domain. A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment thereof. Exemplary costimulatory intracellular signaling domains include those derived from molecules responsible for costimulatory signals (e.g., antigen independent stimulation), and those derived from cytokine receptors. In some embodiments, the one or more intracellular signaling domains comprise a primary intracellular signaling domain, and a costimulatory intracellular signaling domain from one or more co-stimulatory proteins or cytokine receptors.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Examples of such molecules include a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CDl 1a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. For example, CD27 co-stimulation has been demonstrated to enhance expansion, effector function, and survival of human CART cells in vitro and augments human T cell persistence and antitumor activity in vivo (Song et al. Blood. 2012; 119 (3): 696-706). In some embodiments, the co-stimulatory domain of the CARs described herein comprises one or more signaling domains from one or more co-stimulatory protein or cytokine receptor selected from CD28, 4-1BB, 2B4, KIR, CD27, OX40, ICOS, MYD88, IL2 receptor, and SynNotch. In some embodiments, the co-stimulatory domain of the CARs described herein comprises a 4-1BB costimulatory signaling domain. In some embodiments, the 4-1BB co-stimulatory signaling domain comprises the amino acid sequence of: KRGRKKLLYIFKQPFMRPVQTTQEEDGCSCRFPEEEEGGCEL (SEQ ID NO: 12). In some embodiments, the 4-1BB co-stimulatory signaling domain of the CAR described herein comprises an amino acid sequence that is at least 70% identical (e.g., at least 70%, at least 75%, at least 80%, at least 85%, at least 90%, at least 95% or at least 99% identical) to the amino acid sequence of SEQ ID NO: 12.

In some embodiments, the intracellular signaling domain of the CAR described herein comprise the primary signaling domain, e.g., an ITAM containing domain such as a CD3-zeta signaling domain, by itself or combined with a costimulatory signaling domain (e.g., a co-stimulating domain from one or more co-stimulatory protein or cytokine receptor selected from CD28, 4-1BB, 2B4, KIR, CD27, OX40, ICOS, MYD88, IL2 receptor, and SynNotch). In some embodiments, the intracellular signaling domain of the CAR described herein comprise a CD3-zeta (CD32) signaling domain and a 4-1BB costimulatory signaling domain.

In some embodiments, different linker sequences may be used between the different domains of the CAR, e.g., a (GGGS)n (SEQ ID NO: 35) linker, wherein n is 1-20 (e.g., 1, 2, 3, 4, 5, 6, 7, 8, 9, 10, 11, 12, 13, 14, 15, 16, 17, 18, 19, or 20). In some embodiments, the linker is (GGGS)₇ (SEQ ID NO: 36). In some embodiments, the CAR comprises additional sequences from CD27, e.g., the stalk and hinge region of CD27, between the extracellular target binding domain and the transmembrane region. In some embodiments, the stalk and hinge region of CD27 comprises the amino acid sequence of:

PLPNPSLTARSSQALSPHPQPTHLPYVSEMLEARTAGHMQTLADFRQLPARTLSTHW PPQRSLCSSDFIR (SEQ ID NO: 13). In some embodiments, the CAR does not comprise additional sequences from CD27, e.g., the stalk and hinge region of the between the extracellular target binding domain and the transmembrane region.

In some embodiments, the CAR described herein comprises an amino acid sequence that is at least 70% identical (e.g., at least 70%, at least 75%, at least 80%, at least 85%, at least 90%, at least 95% or at least 99% identical) to the amino acid sequence of any one of SEQ ID NOs: 2-7. In some embodiments, the CAR described herein comprises the amino acid sequence of any one of SEQ ID NOs: 2-7.

In some embodiments, the CARs described herein further comprises a leader sequence (also referred herein to as a signal peptide) at the amino-terminus (N-terminus) of the antigen binding domain. In some embodiments, the CAR further comprises a leader sequence at the N-terminus of the antigen binding domain, wherein the leader sequence is optionally cleaved from the antigen binding domain (e.g., a scFv) during cellular processing and localization of the CAR to the cellular membrane. In some embodiments, the leader sequence is a CD27 signal peptide (e.g., a peptide having the amino acid sequence of: MARPHPWWLCVLGTLVGLS (SEQ ID NO: 14)) In some embodiments, the leader sequence is an interleukin 2 signal peptide or a CD8 leader sequence. In some embodiments, the leader sequence comprises an amino acid sequence of: MALPVTALLLPLALLLHAARP (SEQ ID NO: 15).

In some embodiments, the CARs described herein further comprises additional amino acid sequences (e.g., between the extracellular target binding domain and the leader sequence. In some embodiments, the additional sequence is an affinity tag (e.g., a Myc tag, EQKLISEEDL (SEQ ID NO: 16)).

### T-Cell Engaging Antibody Molecule (TEAM)

In some aspects, the present application discloses cells (e.g., T cells) comprising a CAR that binds CD70 (CD70 CAR) (e.g., as described above) and TEAM that binds CD33 (CD33 TEAM). A TEAM, as described herein, is a molecule comprising a first binding moiety (e.g., an anti-CD33 antibody) and a second binding moiety (e.g., an immune cell binding moiety). Without wishing to be bound by theory, the CD33 TEAM helps overcome cancer antigen escape (e.g., the cancer cell no longer expresses the molecule targeted by the CAR) by providing a secondary binding site (i.e., CD33) for engaging immune cells to the cancer. In some embodiments, the CD33 TEAM engages T cells to the cancer. The CD33 TEAM may engage both the CAR-T cell and native T cell to the cancer cells. As disclosed herein, CAR-T cell expressing a CD70 CAR and a CD33 TEAM have increased efficacy in treating relapsed cancer cells that have CD70 loss.

In some embodiments, the CD33 TEAM comprises an anti-CD33 antibody and an immune cell binding moiety. In some embodiments, the anti-CD33 antibody includes but is not limited to a monoclonal antibody, a polyclonal antibody, a recombinant antibody, a human antibody, a humanized antibody, and a functional fragment thereof, including but not limited to a single-chain variable fragment (scFv), a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, the anti-CD33 antibody comprises from N-terminal to C-terminal a VH domain and then a VL domain. In some embodiments, the anti-CD33 antibody comprises from N-terminal to C-terminal a VL domain and then a VH domain. In some embodiments, the CD33 antibody is an scFv. In some embodiments, the CD33 antibody comprises a VL domain of SEQ ID NO: 19 or a variant thereof, and a VH domain of SEQ ID NO: 20 or a variant thereof.

In some embodiments, the anti-CD33 antibody comprises any one of the antibodies or antigen binding domains (e.g., CDRs or VH and VL) as described in U.S. Pat. Nos. 11,136,390, 10,556,951, 10,787,514, 8,759,494, 10,000,566, 11,174,313, 10,711,062, 11,466,082, and US20210317208, each of which is incorporated by reference in it's entirety.

In some embodiments, the immune cell binding moiety is a molecule that binds to a protein expressed on the surface of a T-cell, an NK cell, a dendritic cell, a macrophage, a B cell, a neutrophil, an eosinophil, a basophil, a mast cell, a myeloid-derived suppressor cell, a mesenchymal stem cell, or combinations thereof, or any precursor, derivative, or progenitor cells thereof. In some embodiments, the immune cell binding moiety is a molecule that binds to a protein expressed on the surface of a T Cell (e.g., CD3). In some embodiments, the immune cell binding moiety binds to a cell surface marker of a T Cell. In some embodiments, the immune cell binding moiety is selected from the group consisting of CD3, CD8, CD4, CXCR3, CCR4, GARP, LAP, CD25, CTLA-4, or CD16. In some embodiments, the immune cell binding moiety is an antibody (e.g., an antibody that binds to a protein expressed on the surface of a T-Cell) as described herein. In some embodiments, immune cell binding moiety includes but is not limited to a monoclonal antibody, a polyclonal antibody, a recombinant antibody, a human antibody, a humanized antibody, and a functional fragment thereof, including but not limited to a single-chain variable fragment (scFv), a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some embodiments, the immune cell binding moiety is an scFv. In some embodiments, the immune cell binding moiety is an anti-CD3 antibody. In some embodiments, the anti-CD3 antibody comprises from N-terminal to C-terminal a VH domain and then a VL domain. In some embodiments, the anti-CD33 antibody comprises from N-terminal to C-terminal a VL domain and then a VH domain. In some embodiments, the immune cell binding moiety comprises an amino acid sequence of SEQ ID NO: 21 (anti-CD3 scFv) or a variant thereof.

In some embodiments, the anti-CD3 antibody comprises any one of the antibodies or antigen binding domains (e.g., CDRs or VH and VL) as described in U.S. Pat. No. 9,657,102, US20210244815, U.S. Pat. Nos. 11,530,275, 10,759,858, US20220380464, US20210253701, and U.S. Pat. No. 11,505,606.

In some embodiments, the CD33 TEAM comprises a linker between the CD33 antibody and the immune cell binding moiety. In some embodiments, the linker is a peptide linker. In some embodiments, the linker is non-cleavable. In some embodiments, the linker is a glycine linker. In some embodiments, the linker is a GlySer linker. In some embodiments, the GlySer linker comprises the amino acid sequence GGGS (SEQ ID NO: 34). In some embodiments, the GlySer linker comprises the amino acid sequence (GGGS)2 (SEQ ID NO: 37), (GGGS)3 (SEQ ID NO: 26), (GGGS)4 (SEQ ID NO: 38), (GGGS)5 (SEQ ID NO: 39), (GGGS) 6 (SEQ ID NO: 40). In some embodiments, the GlySer linker comprises the amino acid sequence (GGGGS)2 (SEQ ID NO: 41), (GGGGS)3 (SEQ ID NO: 27), (GGGGS)4 (SEQ ID NO: 28), (GGGGS) 5 (SEQ ID NO: 42), (GGGGS)6 (SEQ ID NO: 43). In some embodiments, the linker comprises an amino acid sequence of any one of SEQ ID NOs: 26-30. In some embodiments, the linker comprises an amino acid sequence of SEQ ID NOs: 26. In some embodiments, the linker is any linker described herein.

In some embodiments, the CD33 TEAM comprises a signal peptide. In some embodiments, the signal peptide is an IgK signal peptide, a CD8 signal peptide, or a CD27 signal peptide. In some embodiments, the signal peptide comprises the amino acid sequence of any one of SEQ ID NOs: 14, 15 or 33, or including the amino acid sequence of any one of SEQ ID NOs: 14, 15 or 33, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of any one of SEQ ID NOs: 14, 15 or 33.

In some embodiments, the CD33 TEAM comprises a CD33 scFv, a non-cleavable linker, and a CD3 scFv. In some embodiments, the CD33 TEAM comprises a CD33 scFv, a non-cleavable linker, a CD3 scFv, a His6 tag, a T2A self-cleavable sequence, and a fluorescent protein (e.g., mCherry).

In some embodiments, the CD33 TEAM is codon-optimized. Codon-optimization is a process of introducing silent mutations into a nucleic acid sequence encoding a protein (e.g., a nucleic acid sequence encoding a CD33 TEAM) that improve the expression of the protein (e.g., the CD33 TEAM). Codon-optimization does not alter the amino acid sequence of the protein, Methods of codon optimization are well known in the art, e.g., as described in Mauro et al., BioDrugs 32.1 (2018): 69-81 and.

In some embodiments, the CD33 TEAM comprises an amino acid sequence that is at least 85% (e.g., at least 85%, at least 90%, at least 95%, at least 96%, at least 97%, at least 98%, at least 99%, or at least 99.5%) identical to any one of SEQ ID NOs: 17-18. In some embodiments, the CD33 TEAM comprises an amino acid sequence of any one of SEQ ID NOs: 17-18. In some embodiments, the CD33 TEAM consists of an amino acid sequence of any one of SEQ ID NOs: 17-18.

### CD70 CAR—CD33 TEAM Constructs

In some embodiments, the CD70 CAR CD33 TEAM construct comprises a CD70 CAR as described herein and a CD33 TEAM as described herein. A construct, as used herein, refers to a nucleic acid sequence or an amino acid sequence that comprises one or more components (e.g. comprises a CD70 CAR and a CD33 TEAM). In some embodiments, the CD70 CAR-CD33 TEAM construct comprises a CD70 CAR comprising CD27 or a fragment thereof that is capable of binding to CD70 as described herein. In some embodiments, the CD70 CAR-CD33 TEAM construct comprises a linker between the CD70 CAR and the CD33 team. In some embodiments, the linker is a cleavable linker. In some embodiments, the cleavable linker is a protease cleavable linker. In some embodiments, the linker is a self-cleavable linker (e.g., P2A (SEQ ID NO: 32), E2A, F2A, or T2A (SEQ ID NO: 31)). In some embodiments, the linker comprises an internal ribosome entry site (IRES). Without being bound to theory, the CD70 CAR CD33 TEAM construct when expressed as a fusion protein may undergo cleavage at a cleavable linker between the CD70 CAR and the CD33 TEAM. This cleavage releases the CD33 TEAM. The CD33 TEAM may be secreted from the cell and may bind to cancer cells expressing CD33 and to immune cells (e.g., CAR-T cells, untransduced T cells, endogenous T cells, and/or bystander T Cells expressing CD3). In some embodiments, the CD33 TEAM may activate CAR-T cells, untransduced T cells, endogenous T cells, and/or bystander T Cells expressing CD3.

In some embodiments, the CD70 CAR and the CD33 TEAM are each operably linked to a promoter, e.g., a promoter as described herein. In some embodiments, the CD70 CAR and the CD33 TEAM are operably linked to the same promoter. In some embodiments, the CD70 CAR and the CD33 TEAM are operably linked to different promotors. In some embodiments, the promoters are constitutive promoters and described herein. In some embodiments, the promoters and inducible promoters and describe herein. In some embodiments, the CD70 CAR CD33 TEAM is operably linked to an EF1alpha promoter.

In some embodiments, the CD70 CAR CD33 TEAM construct comprises from N-terminal to C-terminal a CD70 CAR (e.g. CD27 CAR), a cleavable linker (e.g. P2A), and a CD33 TEAM. In some embodiments, the CD70 CAR CD33 TEAM construct comprises from N-terminal to C-terminal a CD33 TEAM, a cleavable linker (e.g. P2A), and a CD70 CAR (e.g. CD27 CAR). In some embodiments, the CD70 CAR CD33 TEAM comprises a truncated CD27 CAR and a CD33 TEAM. In some embodiments, the CD70 CAR CD33 TEAM comprises from N-terminal to C-Terminal a truncated CD27 (trCD27), a CD8 Hinge, a CD8 transmembrane, a 4-1BB co-stimulatory signaling domain, a CD3-zeta domain, a P2A cleavable peptide, an IgK leader, a CD33 scFv and a CD3 scFv. In some embodiments, the CD70 CAR CD33 TEAM comprises from N-terminal to C-Terminal a truncated CD27 (trCD27), a CD8 Hinge, a CD8 transmembrane, a 4-1BB intracellular signaling domain, a CD3-zeta signaling domain, a P2A cleavable peptide, an IgK leader, a CD33 scFv, a non-cleavable linker, and a CD3 scFv. In some embodiments, the CD70 CAR CD33 TEAM comprises from N-terminal to C-Terminal a truncated CD27 (trCD27), a CD8 Hinge, a CD8 transmembrane, a 4-1BB intracellular signaling domain, a CD3-zeta domain, a P2A cleavable peptide, an IgK leader, a CD33 scFv, a non-cleavable linker, a CD3 scFv, a His6 tag, a T2A cleavable peptide, and a fluorescent protein (e.g., mCherry).

In some embodiments, the CD70 CAR CD33 TEAM construct comprises an amino acid sequence that is at least at least 85% (e.g., at least 85%, at least 90%, at least 95%, at least 96%, at least 97%, at least 98%, at least 99%, or at least 99.5% identical to any one of SEQ ID NOs: 22-25. In some embodiments, the CD70 CAR CD33 TEAM construct comprises an amino acid sequence of any one of SEQ ID NOs: 22-25. In some embodiments, the CD70 CAR CD33 TEAM construct comprises an amino acid sequence of any one of SEQ ID NOs: 22-23. In some embodiments, the CD70 CAR CD33 TEAM construct consists of an amino acid sequence of any one of SEQ ID NOs: 22-25.

In some aspects, the disclosure provides nucleic acid molecules (e.g., vectors) for expressing CD70 CAR CD33 TEAM constructs in cells, e.g., T cells. In some embodiments, the nucleic acid molecule comprises a nucleotide sequence encoding the CD70 CAR CD33 TEAM constructs described herein. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989; and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference). Alternatively, the gene of interest can be produced synthetically, rather than cloned.

In some embodiments, the desired CD70 CAR CD33 TEAM constructs can be expressed in the cells by way of transposons. In some embodiments, expression of natural or synthetic nucleic acids CARs is typically achieved by operably linking a nucleic acid encoding the CAR to a promoter, and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence. The expression constructs of the disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuL V promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure is not limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, the promoter is an EF-1a promoter.

In some embodiments, the nucleic acid comprising a nucleotide sequence encoding the CD70 CAR CD33 TEAM construct described herein is a vector. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. In some embodiments, retrovirus vectors are used. In some embodiments, lentivirus vectors are used. In some embodiments, adeno-associated virus (AAV) vectors can also be used.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Any methods known in the art for delivering nucleic acids or proteins into a cell may be used, e.g., transfection, transformation, transduction, or electroporation. The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

### Cells Comprising a CD70 CAR and CD33 TEAM

In some aspects, this application discloses cells comprising a CD70 CAR and a CD33 TEAM as described herein. In some embodiments, the cell comprises a CD70 CAR-CD33 TEAM construct, as described herein. In some embodiments, the cells are immune cells. In some embodiments, the immune cell is a mammalian immune cell. In some embodiments, the immune cell is a human immune cell. An “immune cell” can be a T-cell, an NK cell, a dendritic cell, a macrophage, a B cell, a neutrophil, an eosinophil, a basophil, a mast cell, a myeloid-derived suppressor cell, a mesenchymal stem cell, or combinations thereof, or any precursor, derivative, or progenitor cells thereof. In some embodiments, the immune cell is a T cell. In some embodiments, the immune cell is a human T cell.

Immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. The immune cells (e.g., T cells) may also be generated from induced pluripotent stem cells or hematopoietic stem cells or progenitor cells. In some embodiments, any number of immune cell lines, including but not limited to T cell lines, including, for example, Hep-2, Jurkat, and Raji cell lines, available in the art, may be used. In some embodiments, immune cells (e.g., T cells) can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, NK cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, immune cells (e.g., T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD1 1b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in some embodiments, T regulatory cells are depleted by anti-C25 conjugated beads or other similar method of selection.

The engineered immune cells (e.g., T cells) may be autologous. Being “autologous” means the immune cells are obtained from a subject, engineered to express a CAR described herein, and administered to the same subject. Administration of autologous cells to a subject may result in reduced rejection of the immune cells as compared to administration of non-autologous cells. Alternatively, the engineered immune cells (e.g., T cells) can be allogeneic cells. Being “allogeneic” the cells are obtained from a first subject, modified to express the CAR described herein and administered to a second subject that is different from the first subject but of the same species. For example, allogeneic immune cells may be derived from a human donor and administered to a human recipient who is different from the donor.

In some embodiments, this application discloses a CAR-T Cell comprising a CD70 CAR (e.g. CD27 extracellular binding domain) and a CD33 TEAM (e.g. comprising an anti-CD33 antibody and an anti-CD3 antibody) as described herein. In some embodiments, the CAR-T cell comprises a CD70 CAR-CD33 TEAM construct encoding an amino acid sequence of any one of SEQ ID NOs: 22-25.

### Methods of Treatment

In some embodiments, this application discloses methods of treating cancer (e.g. cancer characterized by cells expressing CD70 and CD33) comprising administering an effective amount of a cell (e.g. a CAR-T cell comprising a CD70 CAR and a CD33 TEAM) to a subject. In some embodiments, the method is for treating a cancer characterized by cells expressing CD70, and the method comprises administering to a subject in need thereof an effective amount of the engineered immune cells (e.g., CD70 CAR CD33 TEAM CAR-T cells) or the compositions comprising the engineered immune cells (e.g., CD70 CAR CD33 TEAM CAR-T cells) described herein. In some embodiments, the method is for treating a cancer characterized by cells expressing CD70 and cells that express a lower level of CD70 (i.e., lower than the level of the aforementioned cancer cells expressing CD70) or that do not express CD70, and the method comprises administering to a subject in need thereof an effective amount of the engineered immune cells (e.g., CD70 CAR CD33 TEAM CAR-T cells) or the compositions comprising the engineered immune cells (e.g., CD70 CAR CD33 TEAM CAR-T cells) described herein. In some embodiments, the method comprises administering CD70 CAR CD33 TEAM CAR-T cells to a subject having or diagnosed as having a cancer that express CD70. In some embodiments, the method comprises administering CD70 CAR CD33 TEAM CAR-T cells to a subject having cancer cells that express CD70. In some embodiments, the method comprises administering CD70 CAR CD33 TEAM CAR-T cells to a subject having cancer cells that express CD70 and CD33. In some embodiments, the method comprises administering CD70 CAR CD33 TEAM CAR-T cells to a subject having cancer cells that express CD33. In some embodiments, the method comprises administering CD70 CAR CD33 TEAM CAR-T cells to a subject having cancer cells that are resistant to CD70 CAR-based treatments (e.g., CD70 expression is decreased or a mutation decreases CD70 CAR binding to CD70 of the cancer cell). In some embodiments, the method comprises administering CD70 CAR CD33 TEAM CAR-T cells to a subject having cancer cells that have decreased CD70 expression. Decreased CD70 expression, as used herein, is decreased relative to a cancer cell that express CD70 at an amount that is sufficient for CD70 CAR T Cell directed killing of the cancer cell (e.g., a CD70 expressing cancer cell that has not yet been treated with an anti-CD70 therapeutic). In some embodiments, decreased CD70 expression is at least 0.1% (e.g., at least 0.5%, at least 1%, at least 5%, at least 10%, at least 15%, at least 20%, at least 30%, at least 40%, at least 50%, at least 60%, at least 70%, at least 80%, at least 90%, at least 95%, at least 98%, or at least 99%) decreased compared to a CD70 expressing cancer cell that has not yet been treated with an anti-CD70 therapeutic (e.g., a CD70 CAR) and/or a CD70 expressing cancer cell that is expected to be killed by an anti-CD70 CAR T Cell. In some embodiments, decreased CD70 expression is at least 0.1% (e.g., at least 0.5%, at least 1%, at least 5%, at least 10%, at least 15%, at least 20%, at least 30%, at least 40%, at least 50%, at least 60%, at least 70%, at least 80%, at least 90%, at least 95%, at least 98%, or at least 99%) decreased compared to CD70 expression of a Molm13WT cell. In some embodiments, decreased CD70 expression refers to no detectable CD70 expression (e.g., no detectable CD70 cell surface expression).

In some embodiments, the method comprises administering the CD70 CAR CD33 TEAM CAR-T Cells described herein to a subject having a cancer that expresses CD70. The skilled person will understand that after one or administrations, some of the cancer cells may have decreased CD70 expression to avoid being bound by the CD70 CAR CD33 TEAM CAR-T Cells. Thus, in some embodiments, the method of administering the CD70 CAR CD33 TEAM CAR-T Cells described herein to a subject having a cancer that expresses CD70 includes administering one or more rounds of CD70 CAR CD33 TEAM CAR-T Cells to cancers cells that have decreased CD70 expression.

In some embodiments, the method comprises administering a CD70 CAR CAR-T Cell to a subject having a cancer that expresses CD70, and, if the cancer decreases CD70 expression or otherwise becomes resistant to the CD70 CAR-T cells, the method further comprises administering CD70 CAR CD33 TEAM CAR-T Cells.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

In some embodiments, treatment of cancer in a subject using CD70 CAR CD33 TEAM CAR-T Cells does not result systemic toxicity. In some embodiments, treatment of cancer in a subject using CD70 CAR CD33 TEAM CAR-T Cells does not result in CD33 TEAM systemic toxicity. In some embodiments, treatment of cancer in a subject using CD70 CAR CD33 TEAM CAR-T Cells does not result hepatotoxicity. In some embodiments, treatment of cancer in a subject using CD70 CAR CD33 TEAM CAR-T Cells does not result in CD33 TEAM hepatotoxicity.

In some embodiments, the method is for treating a cancer characterized by cells expressing CD70, and the method comprises administering to a subject in need thereof an effective amount of the engineered immune cells (e.g., CD70 CAR CD33 TEAM CAR-T cells) or the compositions comprising the engineered immune cells (e.g., CD70 CAR CD33 TEAM CAR-T cells) described herein, and an effective amount of an agent that enhances expression of CD70 in the cancer (e.g., azacitidine or decitabine).

Examples of cancers characterized by cells that express CD70 include, without limitation, bladder cancer, breast invasive carcinoma, cervical cancer, cholangiocarcinoma, colorectal cancer, diffuse large B-cell lymphoma (DLBC), Esophagus, glioblastoma (GBM), head and neck cancer, low-grade gliomas (LGG), liver cancer, lung adeno cancer, melanoma, mesothelioma, ovarian cancer, pancreatic cancer, prostate cancer, sarcoma, stomach cancer, testicular germ cell cancer, thymoma, thyroid cancer, uterine cancer, uveal melanoma, clear cell renal cell carcinoma (ccRCC), chromophobe renal cell carcinoma, papillary renal cell carcinoma (pRCC), acute myeloid leukemia, and adenoid cystic carcinoma (ACC). In some embodiments, the cancer is a lymphoma. In some embodiments, the lymphoma is a B-cell Non-Hodgkin Lymphoma (NHL), mantle cell lymphoma, Burkitt's lymphoma, B-cell lymphoblastic lymphoma, diffuse large B-cell lymphoma (DLBCL), follicular lymphoma (FL), marginal zone lymphoma, or T-cell lymphoma. In some embodiments, the cancer is a leukemia. In some embodiments, the leukemia is acute myeloid leukemia (AML), small lymphocytic lymphoma (SLL), chronic myeloid leukemia (CML), acute lymphocytic leukemia (ALL), B-cell lymphoblastic leukemia, chronic lymphocytic leukemia (CLL), or T-cell leukemia. In some embodiments, the cancer is a myeloid cancer. In some embodiments, the cancer is acute myeloid leukemia.

Other aspects of the present disclosure provide compositions comprising any one of the immune cells (e.g., CD70 CAR CD33 TEAM CAR-T cells) described herein. In some embodiments, the composition comprising the engineered immune cells (e.g., CD70 CAR CD33 TEAM CAR-T cells) further comprises an agent that enhances CD70 expression in cancer cells. In some embodiments, the agent results in hypomethylation of CD-70 encoding gene in the cancer. In some embodiments, the agent is azacitidine or decitabine. In some embodiments, the composition comprises the engineered immune cells (e.g., CD70 CAR CD33 TEAM CAR-T cells) and azacitidine. In some embodiments, the composition comprises the engineered immune cells (e.g., CD70-targeting CAR-T cells) and azacitidine, wherein azacitidine has a concentration of 100 μM or less (e.g., 100 μM or less, 90 μM or less, 80 μM or less, 70 μM or less, 60 μM or less, 50 μM or less, 40 UM or less, 30 μM or less, 20 μM or less, 10 μM or less, 5 μM or less, 1 μM or less) in the composition. In some embodiments, the composition comprises the engineered immune cells (e.g., CD70 CAR CD33 TEAM CAR-T cells) and azacitidine, wherein azacitidine has a concentration of 100 μM, 90 μM, 80 μM, 70 μM, 60 μM, 50 μM, 40 μM, 30 μM, 20 μM, 10 μM, 5 μM, or 1 μM in the composition.

### Administration

In some embodiments, the composition is a pharmaceutical composition. In some embodiments, the composition further comprises a pharmaceutically acceptable carrier, excipients or stabilizers typically employed in the art (all of which are termed “excipients”), for example buffering agents, stabilizing agents, preservatives, isotonifiers, non-ionic detergents, antioxidants and/or other miscellaneous additives.

In some embodiments, any one of the engineered immune cells (e.g., CD70 CAR CD33 TEAM CAR-T cells) described herein or any one of the compositions comprising the engineered immune cells described herein is administered to a subject. Accordingly, some aspects of the present disclosure provide methods of administering to a subject any one of the engineered immune cells (e.g., CD70 CAR CD33 TEAM CAR-T cells) or the compositions comprising the engineered immune cells (e.g., CD70 CAR CD33 TEAM CAR-T cells) described herein.

In some embodiments, the engineered immune cells (e.g., CD70 CAR CD33 TEAM CAR-T cells) and the agent (e.g., azacitidine or decitabine) are administered simultaneously (e.g., the engineered immune cell and the agent are formulated in a composition for administration). In some embodiments, the composition comprises the engineered immune cells (e.g., CD70-targeting CAR-T cells) and azacitidine, wherein azacitidine has a concentration of 100 μM or less (e.g., 100 μM or less, 90 μM or less, 80 μM or less, 70 μM or less, 60 μM or less, 50 μM or less, 40 μM or less, 30 μM or less, 20 μM or less, 10 μM or less, 5 μM or less, 1 μM or less) in the composition. In some embodiments, the composition comprises the engineered immune cells (e.g., CD70-targeting CAR-T cells) and azacitidine, wherein azacitidine has a concentration of 100 μM, 90 μM, 80 μM, 70 μM, 60 μM s, 50 μM, 40 μM, 30 μM, 20 μM, 10 μM, 5 μM, or 1 μM in the composition.

In some embodiments, the engineered immune cells (e.g., CD70 CAR CD33 TEAM CAR-T cells) and the agent are administered sequentially. In some embodiments, the agent (e.g., azacitidine or decitabine) is administered before the engineered immune cells (e.g., CD70-targeting CAR-T cells) are administered. In some embodiments, there is a waiting period between administering the agent (e.g., azacitidine or decitabine) and administering the engineered immune cell. The waiting period is for the agent (e.g., azacitidine or decitabine) to enhance CD70 expression in the cancer and to clear out of the subject before the engineered immune cells (e.g., CD70 CAR CD33 TEAM CAR-T cells) are administered. In some embodiments, the waiting period is 3 hours or more (e.g., 3, 4, 5, 6, 7, 8, 9, 10, 12, 24 hours or more).

In some embodiments, the agent (e.g., azacitidine or decitabine) enhances CD70 expression in the cancer by at least 10% (e.g., at least 10%, at least 20%, at least 30%, at least 40%, at least 50%, at least 60%, at least 70%, at least 80%, at least 90%, at least 100%, at least 2-fold, at least 5-fold, at least 10-fold, or more), compared to the same cancer without exposure to the agent (e.g., azacitidine or decitabine).

In some embodiments, administering both the engineered immune cells (e.g., CD70-targeting CAR-T cells) and the agent (e.g., azacitidine or decitabine) to the subject enhances the therapeutic efficacy by at least at least 10% (e.g., at least 10%, at least 20%, at least 30%, at least 40%, at least 50%, at least 60%, at least 70%, at least 80%, at least 90%, at least 100%, at least 2-fold, at least 5-fold, at least 10-fold, or more), compared to when the engineered immune cells (e.g., CD70-targeting CAR-T cells) or the agent (e.g., azacitidine or decitabine) is administered alone. Therapeutic efficacy may be measured by methods known in the art, e.g., clearance of cancer cells, prolonged survival of the subject.

To practice the methods described herein, an effective amount of the engineered immune cells (e.g., CD70 CAR CD33 TEAM CAR-T cells) and or the agent that enhances CD70 expression in the cancer (e.g., azacitidine or decitabine) may be administered to a subject via a suitable route (e.g., intravenous infusion). The immune cell population may be mixed with a pharmaceutically acceptable carrier to form a pharmaceutical composition prior to administration, which is also within the scope of the present disclosure.

The subject to be treated may be a mammal (e.g., human, mouse, pig, cow, rat, dog, guinea pig, rabbit, hamster, cat, goat, sheep or monkey). The subject may be suffering from cancer or an immune disorder (e.g., an autoimmune disease).

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The therapeutic methods described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation, or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

TABLE 1
Sequences

| SEQ | | |
|---|---|---|
| ID | | |
| NO | Name | Sequence |
| 1 | CD70 CAR | ATPAPKSCPERHYWAQGKLCCQMCEPGTFLVKDCDQHRKAAQC |
| | extracellular | DPCIPGVSFSPDHHTRPHCESCRHCNSGLLVRNCTITANAECACR |
| | target binding | NGWQCRDKECTECD |
| | domain | |
| 2 | CD70 CAR 1 | ATPAPKSCPERHYWAQGKLCCQMCEPGTFLVKDCDQHRKAAQC |
| | | DPCIPGVSFSPDHHTRPHCESCRHCNSGLLVRNCTITANAECACR |
| | | NGWQCRDKECTECDPLPNPSLTARSSQALSPHPQPTHLPYVSEML |
| | | EARTAGHMQTLADFRQLPARTLSTHWPPQRSLCSSDFIRILVIFSG |
| | | MFLVFTLAGALFLKRGRKKLLYIFKQPFMRPVQTTQEEDGCSCRF |
| | | PEEEEGGCELRVKFSRSADAPAYQQGQNQLYNELNLGRREEYDV |
| | | LDKRRGRDPEMGGKPRRKNPQEGLYNELQKDKMAEAYSEIGMK |
| | | GERRRGKGHDGLYQGLSTATKDTYDALHMQALPPR |
| 3 | CD70 CAR 2 | EQKLISEEDLATPAPKSCPERHYWAQGKLCCQMCEPGTFLVKDC |
| | | DQHRKAAQCDPCIPGVSFSPDHHTRPHCESCRHCNSGLLVRNCTI |
| | | TANAECACRNGWQCRDKECTECDPLPNPSLTARSSQALSPHPQP |
| | | THLPYVSEMLEARTAGHMQTLADFRQLPARTLSTHWPPQRSLCS |
| | | SDFIRILVIFSGMFLVFTLAGALFLKRGRKKLLYIFKQPFMRPVQT |
| | | TQEEDGCSCRFPEEEEGGCELRVKFSRSADAPAYQQGQNQLYNE |
| | | LNLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |
| 4 | CD70 CAR 3 | ATPAPKSCPERHYWAQGKLCCQMCEPGTFLVKDCDQHRKAAQC |
| | | DPCIPGVSFSPDHHTRPHCESCRHCNSGLLVRNCTITANAECACR |
| | | NGWQCRDKECTECDTTTPAPRPPTPAPTIASQPLSLRPEACRPAA |
| | | GGAVHTRGLDFACDIYIWAPLAGTCGVLLLSLVITLYCKRGRKK |
| | | LLYIFKQPFMRPVQTTQEEDGCSCRFPEEEEGGCELRVKFSRSAD |
| | | APAYQQGQNQLYNELNLGRREEYDVLDKRRGRDPEMGGKPRRK |
| | | NPQEGLYNELQKDKMAEAYSEIGMKGERRRGKGHDGLYQGLST |
| | | ATKDTYDALHMQALPPR |
| 5 | CD70 CAR 4 | ATPAPKSCPERHYWAQGKLCCQMCEPGTFLVKDCDQHRKAAQC |
| | | DPCIPGVSFSPDHHTRPHCESCRHCNSGLLVRNCTITANAECACR |
| | | NGWQCRDKECTECDGGGSGGGSGGGSGGGSGGGSGGGSGGGS |
| | | GGGSGGGSGGGSGGGSGGGSGGGSGGGSGGGSGGGSGGGSILVI |
| | | FSGMFLVFTLAGALFLKRGRKKLLYIFKQPFMRPVQTTQEEDGCS |
| | | CRFPEEEEGGCELRVKFSRSADAPAYQQGQNQLYNELNLGRREE |
| | | YDVLDKRRGRDPEMGGKPRRKNPQEGLYNELQKDKMAEAYSEI |
| | | GMKGERRRGKGHDGLYQGLSTATKDTYDALHMQALPPR |
| 6 | CD70 CAR 5 | ATPAPKSCPERHYWAQGKLCCQMCEPGTFLVKDCDQHRKAAQC |
| | | DPCIPGVSFSPDHHTRPHCESCRHCNSGLLVRNCTITANAECACR |
| | | NGWQCRDKECTECDPLPNPSLTARSSLCSSDFIRILVIFSGMFLVF |
| | | TLAGALFLKRGRKKLLYIFKQPFMRPVQTTQEEDGCSCRFPEEEE |
| | | GGCELRVKFSRSADAPAYQQGQNQLYNELNLGRREEYDVLDKR |
| | | RGRDPEMGGKPRRKNPQEGLYNELQKDKMAEAYSEIGMKGERR |
| | | RGKGHDGLYQGLSTATKDTYDALHMQALPPR |
| 7 | CD70 CAR 6 | ATPAPKSCPERHYWAQGKLCCQMCEPGTFLVKDCDQHRKAAQC |
| | | DPCIPGVSFSPDHHTRPHCESCRHCNSGLLVRNCTITANAECACR |
| | | NGWQCRDKECTECDILVIFSGMFLVFTLAGALFLKRGRKKLLYIF |
| | | KQPFMRPVQTTQEEDGCSCRFPEEEEGGCELRVKFSRSADAPAY |
| | | QQGQNQLYNELNLGRREEYDVLDKRRGRDPEMGGKPRRKNPQE |
| | | GLYNELQKDKMAEAYSEIGMKGERRRGKGHDGLYQGLSTATKD |
| | | TYDALHMQALPPR |
| 8 | CD27 | MARPHPWWLCVLGTLVGLSATPAPKSCPERHYWAQGKLCCQM |
| | | CEPGTFLVKDCDQHRKAAQCDPCIPGVSFSPDHHTRPHCESCRHC |
| | | NSGLLVRNCTITANAECACRNGWQCRDKECTECDPLPNPSLTAR |
| | | SSQALSPHPQPTHLPYVSEMLEARTAGHMQTLADFRQLPARTLST |
| | | HWPPQRSLCSSDFIRILVIFSGMFLVFTLAGALFLHQRRKYRSNKG |
| | | ESPVEPAEPCHYSCPREEEGSTIPIQEDYRKPEPACSP |
| 9 | CD27 CD70 | TRPHCESCRHCN |
| | binding domain | |
| 10 | CD27 | ILVIFSGMFLVFTLAGALFL |
| | transmembrane | |
| | domain | |
| 11 | CD3-zeta | RVKFSRSADAPAYQQGQNQLYNELNLGRREEYDVLDKRRGRDP |
| | signaling | EMGGKPRRKNPQEGLYNELQKDKMAEAYSEIGMKGERRRGKG |
| | domain | HDGLYQGLSTATKDTYDALHMQALPPR |
| 12 | 4-1BB co- | KRGRKKLLYIFKQPFMRPVQTTQEEDGCSCRFPEEEEGGCEL |
| | stimulatory | |
| | signaling | |
| | domain | |
| 13 | CD27 Stalk and | PLPNPSLTARSSQALSPHPQPTHLPYVSEMLEARTAGHMQTLADF |
| | hinge | RQLPARTLSTHWPPQRSLCSSDFIR |
| 14 | CD27 signal | MARPHPWWLCVLGTLVGLS |
| | peptide | |
| 15 | CD8 leader | MALPVTALLLPLALLLHAARP |
| | sequence | |
| 16 | Myc tag | EQKLISEEDL |
| 17 | CD33 (VL/VH) | DIQLTQSPSTLSASVGDRVTITCRASESLDNYGIRFLTWFQQKPGK |
| | TEAM | APKLLMYAASNQGSGVPSRFSGSGSGTEFTLTISSLQPDDFATYY |
| | | CQQTKEVPWSFGQGTKVEVKGGGGSGGGGSGGGGSEVQLVQSG |
| | | AEVKKPGSSVKVSCKASGYTITDSNIHWVRQAPGQSLEWIGYIYP |
| | | YNGGTDYNQKFKNRATLTVDNPTNTAYMELSSLRSEDTAFYYC |
| | | VNGNPWLAYWGQGTLVTVSSGGGGSDIKLQQSGAELARPGASV |
| | | KMSCKTSGYTFTRYTMHWVKQRPGQGLEWIGYINPSRGYTNYN |
| | | QKFKDKATLTTDKSSSTAYMQLSSLTSEDSAVYYCARYYDDHYC |
| | | LDYWGQGTTLTVSSVEGGSGGSGGSGGSGGVDDIQLTQSPAIMS |
| | | ASPGEKVTMTCRASSSVSYMNWYQQKSGTSPKRWIYDTSKVAS |
| | | GVPYRFSGSGSGTSYSLTISSMEAEDAATYYCQQWSSNPLTFGAG |
| | | TKLELK |
| 18 | CD33 (VH/VL) | EVQLVQSGAEVKKPGSSVKVSCKASGYTITDSNIHWVRQAPGQS |
| | TEAM | LEWIGYIYPYNGGTDYNQKFKNRATLTVDNPTNTAYMELSSLRS |
| | | EDTAFYYCVNGNPWLAYWGQGTLVTVSSGGGGSGGGGSGGGG |
| | | SDIQLTQSPSTLSASVGDRVTITCRASESLDNYGIRFLTWFQQKPG |
| | | KAPKLLMYAASNQGSGVPSRFSGSGSGTEFTLTISSLQPDDFATY |
| | | YCQQTKEVPWSFGQGTKVEVKGGGGSDIKLQQSGAELARPGAS |
| | | VKMSCKTSGYTFTRYTMHWVKQRPGQGLEWIGYINPSRGYTNY |
| | | NQKFKDKATLTTDKSSSTAYMQLSSLTSEDSAVYYCARYYDDH |
| | | YCLDYWGQGTTLTVSSVEGGSGGSGGSGGSGGVDDIQLTQSPAI |
| | | MSASPGEKVTMTCRASSSVSYMNWYQQKSGTSPKRWIYDTSKV |
| | | ASGVPYRFSGSGSGTSYSLTISSMEAEDAATYYCQQWSSNPLTFG |
| | | AGTKLELK |
| 19 | anti-CD33 scFv | DIQLTQSPSTLSASVGDRVTITCRASESLDNYGIRFLTWFQQKPGK |
| | VL | APKLLMYAASNQGSGVPSRFSGSGSGTEFTLTISSLQPDDFATYY |
| | | CQQTKEVPWSFGQGTKVEVK |
| 20 | anti-CD33 scFv | EVQLVQSGAEVKKPGSSVKVSCKASGYTITDSNIHWVRQAPGQS |
| | VH | LEWIGYIYPYNGGTDYNQKFKNRATLTVDNPTNTAYMELSSLRS |
| | | EDTAFYYCVNGNPWLAYWGQGTLVTVSS |
| 21 | anti-CD3 scFv | DIKLQQSGAELARPGASVKMSCKTSGYTFTRYTMHWVKQRPGQ |
| | | GLEWIGYINPSRGYTNYNQKFKDKATLTTDKSSSTAYMQLSSLTS |
| | | EDSAVYYCARYYDDHYCLDYWGQGTTLTVSSVEGGSGGSGGSG |
| | | GSGGVDDIQLTQSPAIMSASPGEKVTMTCRASSSVSYMNWYQQK |
| | | SGTSPKRWIYDTSKVASGVPYRFSGSGSGTSYSLTISSMEAEDAA |
| | | TYYCQQWSSNPLTFGAGTKLELKHHHHHH |
| 22 | CD27 CAR- | MARPHPWWLCVLGTLVGLSATPAPKSCPERHYWAQGKLCCQM |
| | CD33 TEAM | CEPGTFLVKDCDQHRKAAQCDPCIPGVSFSPDHHTRPHCESCRHC |
| | (VH/VL) | NSGLLVRNCTITANAECACRNGWQCRDKECTECDTTTPAPRPPTP |
| | [CD27-signal- | APTIASQPLSLRPEACRPAAGGAVHTRGLDFACDIYIWAPLAGTC |
| | trCD27- | GVLLLSLVITLYCKRGRKKLLYIFKQPFMRPVQTTQEEDGCSCRF |
| | CD8H/TM-4- | PEEEEGGCELRVKFSRSADAPAYQQGQNQLYNELNLGRREEYDV |
| | 1BBz-CD33H- | LDKRRGRDPEMGGKPRRKNPQEGLYNELQKDKMAEAYSEIGMK |
| | CD33L-CD3 | GERRRGKGHDGLYQGLSTATKDTYDALHMQALPPRSGGSGATN |
| | CAR-TEAM] | FSLLKQAGDVEENPGPMHMETDTLLLWVLLLWVPGSTGDEVQL |
| | | VQSGAEVKKPGSSVKVSCKASGYTITDSNIHWVRQAPGQSLEWI |
| | | GYIYPYNGGTDYNQKFKNRATLTVDNPTNTAYMELSSLRSEDTA |
| | | FYYCVNGNPWLAYWGQGTLVTVSSGGGGSGGGGSGGGGSDIQL |
| | | TQSPSTLSASVGDRVTITCRASESLDNYGIRFLTWFQQKPGKAPK |
| | | LLMYAASNQGSGVPSRFSGSGSGTEFTLTISSLQPDDFATYYCQQ |
| | | TKEVPWSFGQGTKVEVKGGGGSDIKLQQSGAELARPGASVKMS |
| | | CKTSGYTFTRYTMHWVKQRPGQGLEWIGYINPSRGYTNYNQKF |
| | | KDKATLTTDKSSSTAYMQLSSLTSEDSAVYYCARYYDDHYCLD |
| | | YWGQGTTLTVSSVEGGSGGSGGSGGSGGVDDIQLTQSPAIMSAS |
| | | PGEKVTMTCRASSSVSYMNWYQQKSGTSPKRWIYDTSKVASGV |
| | | PYRFSGSGSGTSYSLTISSMEAEDAATYYCQQWSSNPLTFGAGTK |
| | | LELK |
| 23 | CD27 CAR- | MARPHPWWLCVLGTLVGLSATPAPKSCPERHYWAQGKLCCQM |
| | CD33 TEAM | CEPGTFLVKDCDQHRKAAQCDPCIPGVSFSPDHHTRPHCESCRHC |
| | (VL/VH) | NSGLLVRNCTITANAECACRNGWQCRDKECTECDTTTPAPRPPTP |
| | [CD27-signal- | APTIASQPLSLRPEACRPAAGGAVHTRGLDFACDIYIWAPLAGTC |
| | trCD27- | GVLLLSLVITLYCKRGRKKLLYIFKQPFMRPVQTTQEEDGCSCRF |
| | CD8H/TM-4- | PEEEEGGCELRVKFSRSADAPAYQQGQNQLYNELNLGRREEYDV |
| | 1BBz-CD33L- | LDKRRGRDPEMGGKPRRKNPQEGLYNELQKDKMAEAYSEIGMK |
| | CD33H-CD3 | GERRRGKGHDGLYQGLSTATKDTYDALHMQALPPRSGGSGATN |
| | CAR-TEAM] | FSLLKQAGDVEENPGPMHMETDTLLLWVLLLWVPGSTGDDIQLT |
| | | QSPSTLSASVGDRVTITCRASESLDNYGIRFLTWFQQKPGKAPKL |
| | | LMYAASNQGSGVPSRFSGSGSGTEFTLTISSLQPDDFATYYCQQT |
| | | KEVPWSFGQGTKVEVKGGGGSGGGGSGGGGSEVQLVQSGAEVK |
| | | KPGSSVKVSCKASGYTITDSNIHWVRQAPGQSLEWIGYIYPYNGG |
| | | TDYNQKFKNRATLTVDNPTNTAYMELSSLRSEDTAFYYCVNGNP |
| | | WLAYWGQGTLVTVSSGGGGSDIKLQQSGAELARPGASVKMSCK |
| | | TSGYTFTRYTMHWVKQRPGQGLEWIGYINPSRGYTNYNQKFKD |
| | | KATLTTDKSSSTAYMQLSSLTSEDSAVYYCARYYDDHYCLDYW |
| | | GQGTTLTVSSVEGGSGGSGGSGGSGGVDDIQLTQSPAIMSASPGE |
| | | KVTMTCRASSSVSYMNWYQQKSGTSPKRWIYDTSKVASGVPYR |
| | | FSGSGSGTSYSLTISSMEAEDAATYYCQQWSSNPLTFGAGTKLEL |
| | | K |
| 24 | CD27 CAR- | MARPHPWWLCVLGTLVGLSATPAPKSCPERHYWAQGKLCCQM |
| | CD33 TEAM | CEPGTFLVKDCDQHRKAAQCDPCIPGVSFSPDHHTRPHCESCRHC |
| | (VH/VL) | NSGLLVRNCTITANAECACRNGWQCRDKECTECDPLPNPSLTAR |
| | [CD27-signal- | SSQALSPHPQPTHLPYVSEMLEARTAGHMQTLADFRQLPARTLST |
| | trCD27- | HWPPQRSLCSSDFIRILVIFSGMFLVFTLAGALFLKRGRKKLLYIF |
| | CD8H/TM-4- | KQPFMRPVQTTQEEDGCCRFPEEEEGGCELRVKFSRSADAPAY |
| | 1BBz-CD33H- | QQGQNQLYNELNLGRREEYDVLDKRRGRDPEMGGKPRRKNPQE |
| | CD33L-CD3 | GLYNELQKDKMAEAYSEIGMKGERRRGKGHDGLYQGLSTATKD |
| | CAR-TEAM | TYDALHMQALPPRSGGSGATNFSLLKQAGDVEENPGPMHMETD |
| | (with mCherry)] | TLLLWVLLLWVPGSTGDEVQLVQSGAEVKKPGSSVKVSCKASG |
| | | YTITDSNIHWVRQAPGQSLEWIGYIYPYNGGTDYNQKFKNRATL |
| | | TVDNPTNTAYMELSSLRSEDTAFYYCVNGNPWLAYWGQGTLVT |
| | | VSSGGGGSGGGGSGGGGSDIQLTQSPSTLSASVGDRVTITCRASE |
| | | SLDNYGIRFLTWFQQKPGKAPKLLMYAASNQGSGVPSRFSGSGS |
| | | GTEFTLTISSLQPDDFATYYCQQTKEVPWSFGQGTKVEVKGGGG |
| | | SDIKLQQSGAELARPGASVKMSCKTSGYTFTRYTMHWVKQRPG |
| | | QGLEWIGYINPSRGYTNYNQKFKDKATLTTDKSSSTAYMQLSSL |
| | | TSEDSAVYYCARYYDDHYCLDYWGQGTTLTVSSVEGGSGGSGG |
| | | SGGSGGVDDIQLTQSPAIMSASPGEKVTMTCRASSSVSYMNWYQ |
| | | QKSGTSPKRWIYDTSKVASGVPYRFSGSGSGTSYSLTISSMEAED |
| | | AATYYCQQWSSNPLTFGAGTKLELKHHHHHH |
| 25 | CD27 CAR- | MARPHPWWLCVLGTLVGLSATPAPKSCPERHYWAQGKLCCQM |
| | CD33 TEAM | CEPGTFLVKDCDQHRKAAQCDPCIPGVSFSPDHHTRPHCESCRHC |
| | (VL/VH) | NSGLLVRNCTITANAECACRNGWQCRDKECTECDPLPNPSLTAR |
| | [CD27-signal- | SSQALSPHPQPTHLPYVSEMLEARTAGHMQTLADFRQLPARTLST |
| | trCD27- | HWPPQRSLCSSDFIRILVIFSGMFLVFTLAGALFLKRGRKKLLYIF |
| | CD8H&TM-4- | KQPFMRPVQTTQEEDGCSCRFPEEEEGGCELRVKFSRSADAPAY |
| | 1BBz-CD33L- | QQGQNQLYNELNLGRREEYDVLDKRRGRDPEMGGKPRRKNPQE |
| | CD33H-CD3 | GLYNELQKDKMAEAYSEIGMKGERRRGKGHDGLYQGLSTATKD |
| | CAR-TEAM | TYDALHMQALPPRSGGSGATNFSLLKQAGDVEENPGPMHMETD |
| | (with mCherry)] | TLLLWVLLLWVPGSTGDDIQLTQSPSTLSASVGDRVTITCRASESL |
| | | DNYGIRFLTWFQQKPGKAPKLLMYAASNQGSGVPSRFSGSGSGT |
| | | EFTLTISSLQPDDFATYYCQQTKEVPWSFGQGTKVEVKGGGGSG |
| | | GGGSGGGGSEVQLVQSGAEVKKPGSSVKVSCKASGYTITDSNIH |
| | | WVRQAPGQSLEWIGYIYPYNGGTDYNQKFKNRATLTVDNPTNT |
| | | AYMELSSLRSEDTAFYYCVNGNPWLAYWGQGTLVTVSSGGGGS |
| | | DIKLQQSGAELARPGASVKMSCKTSGYTFTRYTMHWVKQRPGQ |
| | | GLEWIGYINPSRGYTNYNQKFKDKATLTTDKSSSTAYMQLSSLTS |
| | | EDSAVYYCARYYDDHYCLDYWGQGTTLTVSSVEGGSGGSGGSG |
| | | GSGGVDDIQLTQSPAIMSASPGEKVTMTCRASSSVSYMNWYQQK |
| | | SGTSPKRWIYDTSKVASGVPYRFSGSGSGTSYSLTISSMEAEDAA |
| | | TYYCQQWSSNPLTFGAGTKLELKHHHHHH |
| 26 | linker-1 (G3S)3 | GGGSGGGSGGGS |
| 27 | linker-2 (G4S)3 | GGGGSGGGGSGGGGS |
| 28 | linker-3 (G4S)4 | GGGGSGGGGSGGGGSGGGGS |
| 29 | linker-4 | GSTSGSGKPGSGEGSTKG |
| 30 | linker-5 | GGSSRSSSSGGGGSGGGG |
| 31 | T2A | SGGGGEGRGSLLTCGDVEENPGPR |
| 32 | P2A | GSGATNFSLLKQAGDVEENPGP |
| 33 | IgK-Signal | METDTLLLWVLLLWVPGSTGD |
| | Sequence | |
| 34 | linker | GGGS |
| 35 | linker-(G3S)20 | GGGSGGGSGGGSGGGSGGGSGGGSGGGSGGGSGGGSGGGSGGG |
| | | SGGGSGGGSGGGSGGGSGGGSGGGSGGGSGGGSGGGS |
| 36 | linker-(G3S)7 | GGGSGGGSGGGSGGGSGGGSGGGSGGGS |
| 37 | linker-(G3S)2 | GGGSGGGS |
| 38 | linker-(G3S)4 | GGGSGGGSGGGSGGGS |
| 39 | linker-(G3S)5 | GGGSGGGSGGGSGGGSGGGS |
| 40 | linker-(G3S)6 | GGGSGGGSGGGSGGGSGGGSGGGS |
| 41 | linker-(G4S)2 | GGGGSGGGGS |
| 42 | linker-(G4S)5 | GGGGSGGGGSGGGGSGGGGSGGGGS |
| 43 | linker-(G4S)6 | GGGGSGGGGSGGGGSGGGGSGGGGSGGGGS |

### EXAMPLES

Acute myeloid leukemia (AML) is the most common acute leukemia in adults and, while uniformly fatal half a century ago, intensive chemotherapy is now curative in forty percent of cases of adults¹. While this represents a substantial improvement, there remains a significant unmet clinical need for older and relapsed/refractory patients where cure rates rapidly fall below ten percent¹. Treatment of AML had changed little over fifty years since the advent of intensive “induction” cytotoxic chemotherapy, however, since 2017 there have been eight FDA drug approvals including for inhibitors of hedgehog, BCL-2, FLT3, IDH1/2, a CD33 antibody drug conjugate, as well as a more potent liposomal formulation of induction chemotherapy². While these interventions represent substantial progress, the majority of AML patients still fail to respond or relapse and die from their disease.

The immunotherapy revolution has seen dramatic responses in a number of malignancies³. However, this modality of therapy has had limited success in AML, possibly owing to its low tumor mutational burden resulting in a dearth of neoantigens, coupled with an immunosuppressive microenvironment with an abundance of myeloid derived suppressor cells (MDSCs), T-regulatory cells (T_{regs}), and exhausted effector (T_{eff}) cells⁴⁻⁸. A recent study of the T-cell subsets and expression of immune checkpoints in patients with newly diagnosed and relapsed AML identified an enrichment of T_{regs} and exhausted T_{eff} cells in AML patients compared to healthy controls.⁹

One way to bypass T cell priming and the limited natural TCR repertoire is to redirect T cells with chimeric antigen receptors (CARs) and infuse CAR-T cells as adoptive immunotherapy. CAR-T cell therapy has rapidly revolutionized the treatment of lymphoid malignancies with two FDA approvals in 2017 for aggressive B-cell malignancies^{10,11} and now four total. These therapies result in destruction of malignant clones, however, there is also an on target, off tumor effect resulting in the elimination of normal B-cells, causing hypogammaglobulinemia which, fortunately, is manageable via administration of intravenous immunoglobulin.

Use of adoptive cellular therapy to treat AML has been more difficult. The majority of the available surface antigens present on AML blasts also reside on many myeloid and stem cell populations, the prolonged ablation of which is not compatible with survival. CARs targeting many different antigens in AML have been described recently (CD123^{12,13}, CD33^{14,15}, FLT3¹⁶), some of which are currently in phase I clinical trials, though none have been as ideal as CD19 has been for lymphoid malignancies¹⁷. At least one of these has led to severe side effects including a death in the first patient treated, possibly due to side effects resulting from CAR targeting of CD123 on normal vasculature¹⁸.

CD70 is a tumor necrosis alpha family member that serves as the ligand for CD27 which is involved in T-cell signaling. CD70 has an extremely restricted expression on normal tissues but marked overexpression in a number of cancer types, including AML^{19,20}. The first AML drug targeting CD70, ARGX-110, an antibody drug conjugate (ADC), has shown impressive response rates in a phase I trial of newly diagnosed AML patients not fit for traditional therapy²¹. Prior, albeit limited, experience with the CD19-targeted bispecific T-cell engager, blinatumomab, suggests that, while effective, CAR-T cells may have enhanced clinical efficacy over traditional antibody based therapies²².

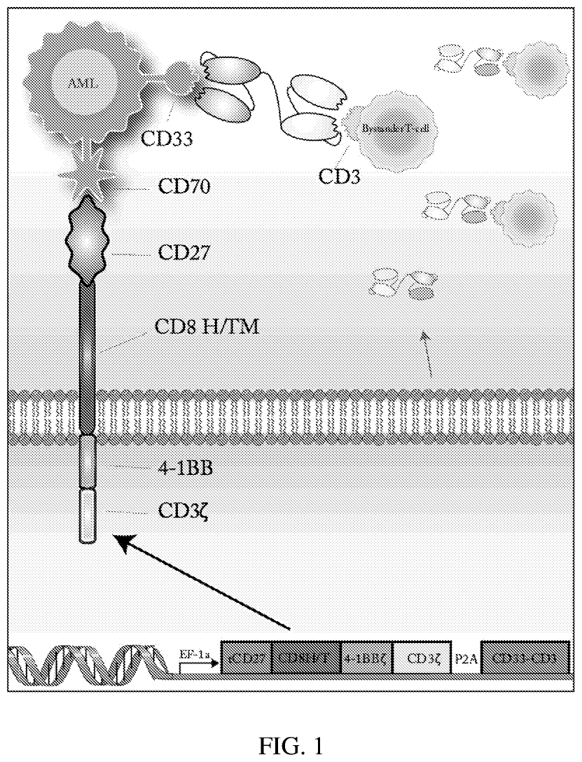

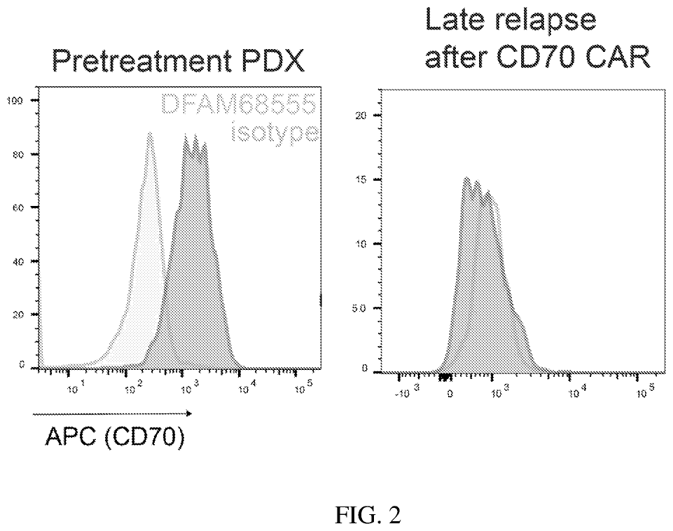

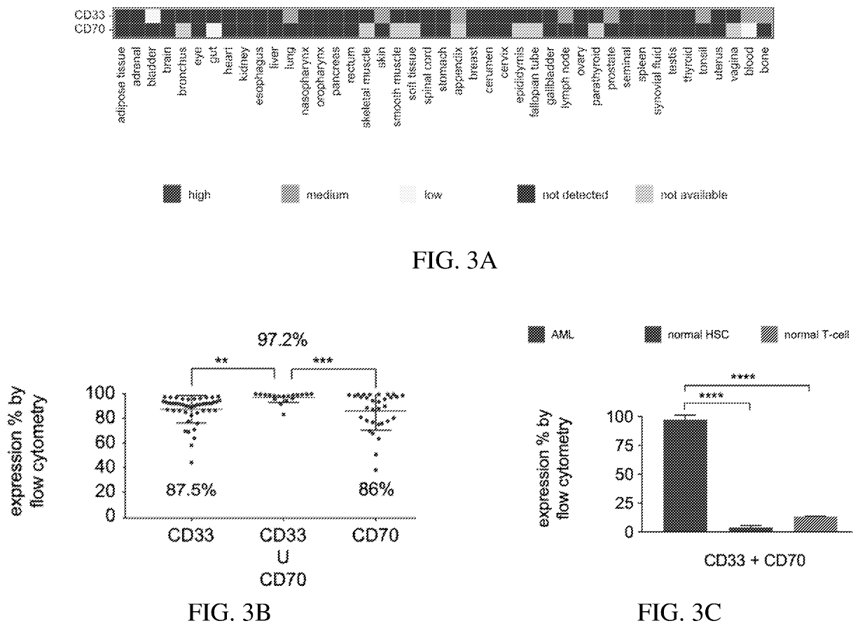

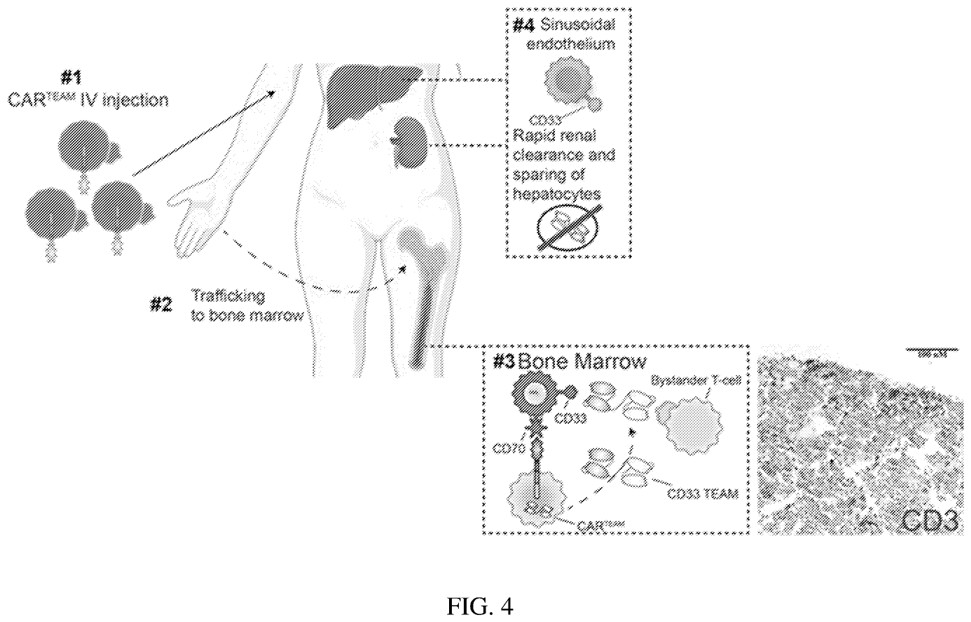

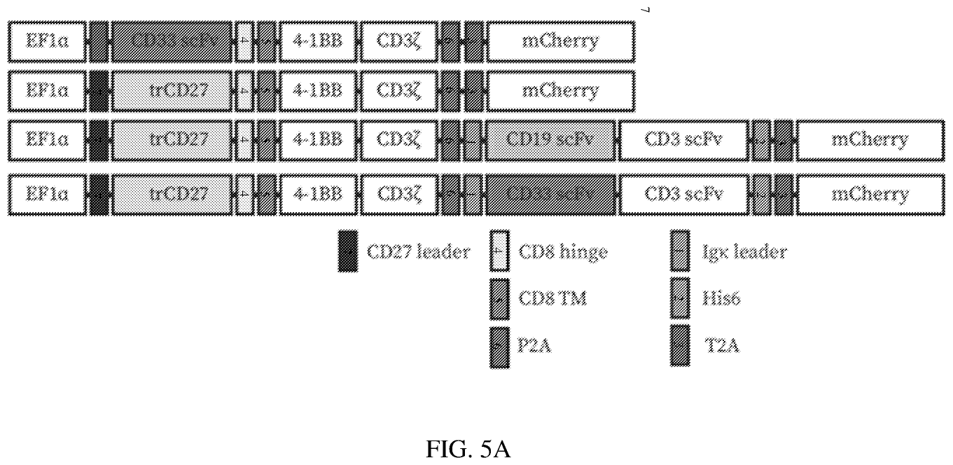

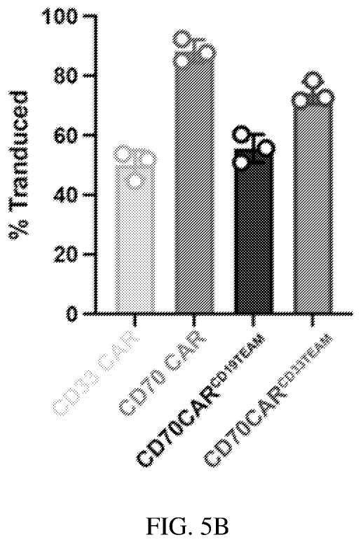

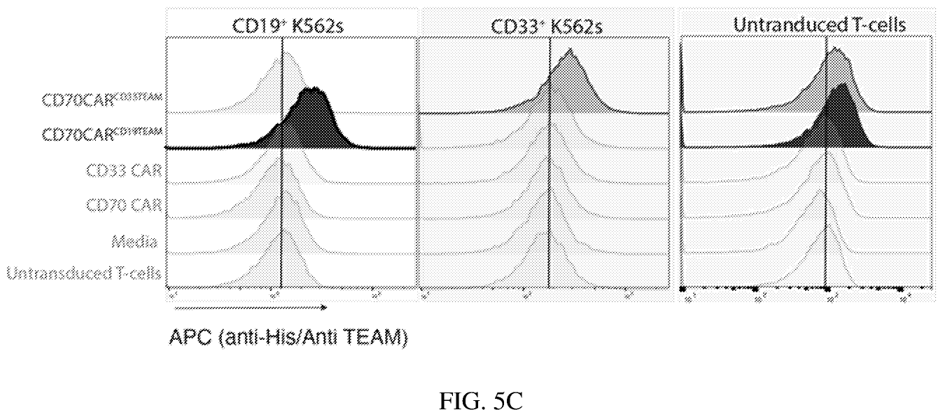

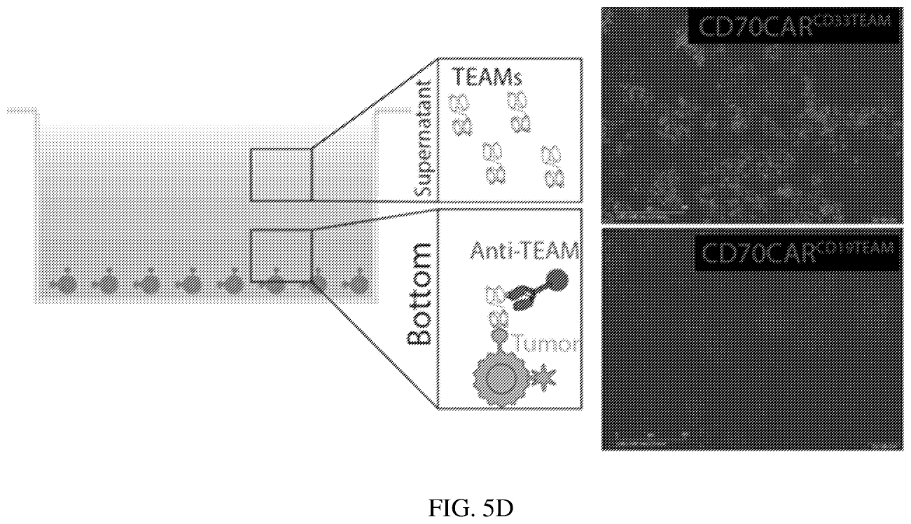

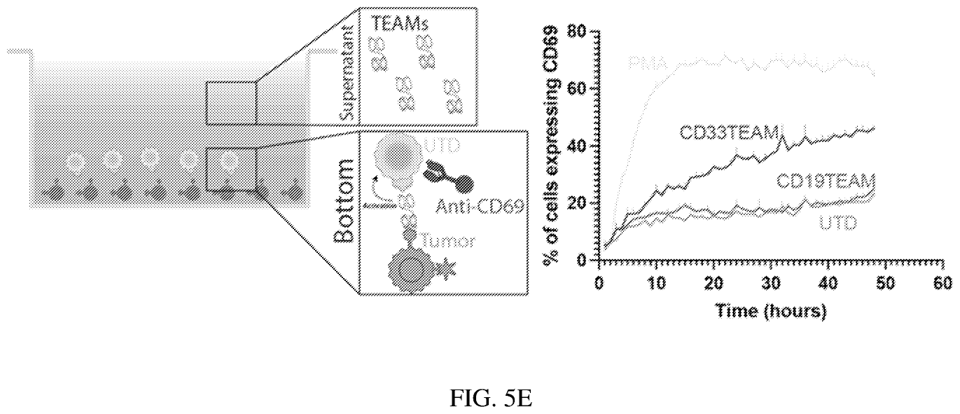

With improvements in clinical management of CAR-T toxicity, the critical problem now facing CAR-T cell therapies for lymphoid malignancies is relapse (FIG. 2). Particularly in B-cell acute lymphoblastic leukemia (ALL), target antigen loss may occur in up to 94% of patients who relapse²³. Soberingly, even modest decreases in well-chosen target antigen expression may be sufficient to evade CAR killing²⁴. To address challenges in antigen escape, several strategies have been employed: bispecfic CARs targeting two antigens with linked single chain variable fragments (tandem CAR), or two entirely separate CARs expressed in the same T-cell (bicistronic CAR) are currently in early phase clinical trials with variable success²⁵⁻²⁹. We have recently pioneered another novel dual targeting strategy utilizing bispecific T-cell engaging antibody molecule (TEAM) secreting CAR-T cells (CAR-TEAM) which simultaneously target multiple antigens and minimize on target toxicity via local TEAM secretion limited to the site of the tumor³⁰. This technique has the added advantage of leveraging the non-transduced—but infused—T-cells representing typically >70% of all infused cells for clinical grade CAR-T cell products (due to the need for low MOIs to preclude multiple viral integration events) via the TEAM secreted from the CAR. Particularly in AML, the lack of uniform expression of any single unique antigen suggests that strategies to increase target antigen expression or a multitargeted approach may be prudent¹⁹. One particularly well characterized AML antigen is CD33 which is expressed in up to 90% of leukemic blasts, (also on normal myeloid cells and some progenitors but not CD34⁺ stem cells³¹)³². Comprehensive AML surfaceome analysis of AML cell lines and primary patient samples projected that simultaneous targeting of CD33 and CD70 in AML would be feasible in over 97% of patients with non-overlapping bystander tissue toxicities¹⁹ (FIGS. 3A-3C). To simultaneously address antigenic heterogeneity, promote local anti-tumor activity through the recruitment of T_{conventional}, T_{reg} and exhausted T_{eff}, and eliminate immunosuppressive MDSCs, a CD3/CD33 TEAM secreting CD70 targeted CAR (CAR-TEAM) was generated (FIG. 1 and FIG. 5A).

The CD70 targeted CAR-T cells traffic to the bone marrow where they lyse AML blasts and simultaneously locally deliver extremely small quantities of a CD33 TEAM to engage T_{conventional} cells and further potentiate the action of CD70 targeted CARs against AML blasts and MDSCs, but not cause systemic toxicity due to extremely rapid clearance of TEAMs in humans²² (FIG. 4).

Despite the larger size of the bicistronic vector, the trCD27-CAR-CD33-TEAM had comparable transduction efficiency to monotargeted constructs with greater than 60% across three healthy donors' T-cells (FIG. 5B). Results show that TEAMs are secreted by CAR-T cells (FIG. 5C). Results also show that TEAMs from the supernatant from the trCD27-CAR-CD33-TEAM bind specifically to target-expressing cells (FIG. 5D). Results also showed that the CD33 TEAM decrease the percent of cells expression CD69 (FIG. 5E).

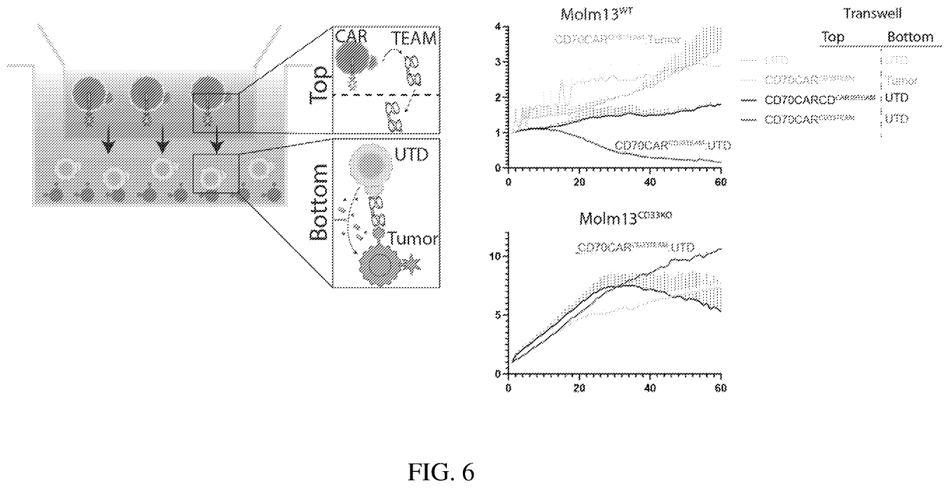

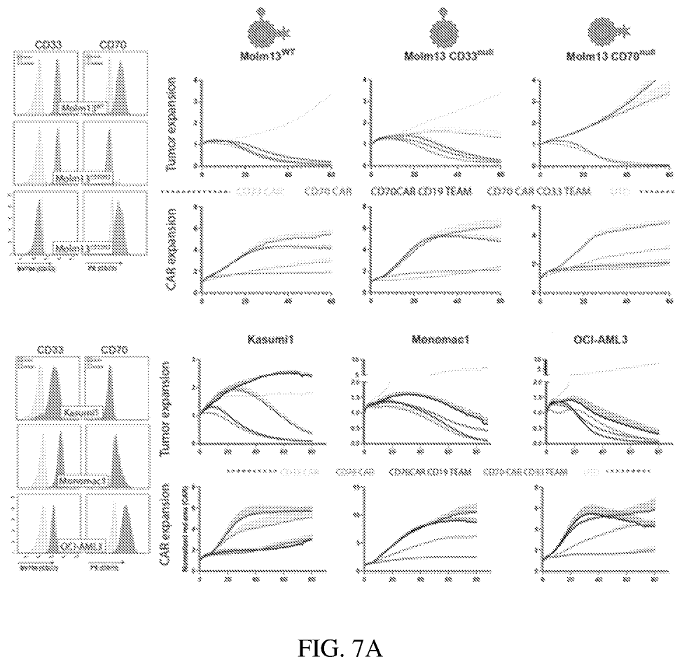

Additional experiments compared the trCD27-CD33-TEAM and trCD27-CD19-TEAM construct in treating Molm13 cells that (1) express CD33, (2) do not express CD33, and (3) do not express CD70. Cells transduced with the CD70CAR^{CD33TEAM} or CD70CAR^{CD19TEAM} constructs, or untransduced T-cells (UTD) were added to the top of a transwell insert. Untransduced T-cells were added with Molm13^{WT} or Molm13^{CD33KO} cells to the bottom of the transwell insert. Only the CD70CAR^{CD33TEAM} construct was able to mediate Molm13^{WT} cell clearance, which was target dependent and was abrogated with loss of CD33. Wells lacking UTD on the bottom were also unable to kill tumor, demonstrating that killing was mediated by the UTDs +TEAMs rather than the TEAM alone. (FIG. 6). These results show TEAMs mediate UTD-driven, target-dependent cytotoxicity. Experiments also showed that simultaneous T-cell redirection through CARs and TEAMs was efficacious against heterogeneous tumors and outperform a mix of individual CARs. Individually targeted CD70 or CD33 CAR-T cells were compared to CD70CAR^{CD33TEAM} or CD70CAR^{CD19TEAM}s against AML targets with different levels of CD70 or CD33 (FIG. 7A, left). CD70CAR^{CD33TEAM} cells exhibited superior cytotoxicity and CAR expansion in this real-time cytotoxicity assay on the incucyte at a 2:1 effector to target ratio. (FIGS. 7A-7B). CAR-T cells were manufactured from 3 healthy donors' T-cells performed in duplicate.

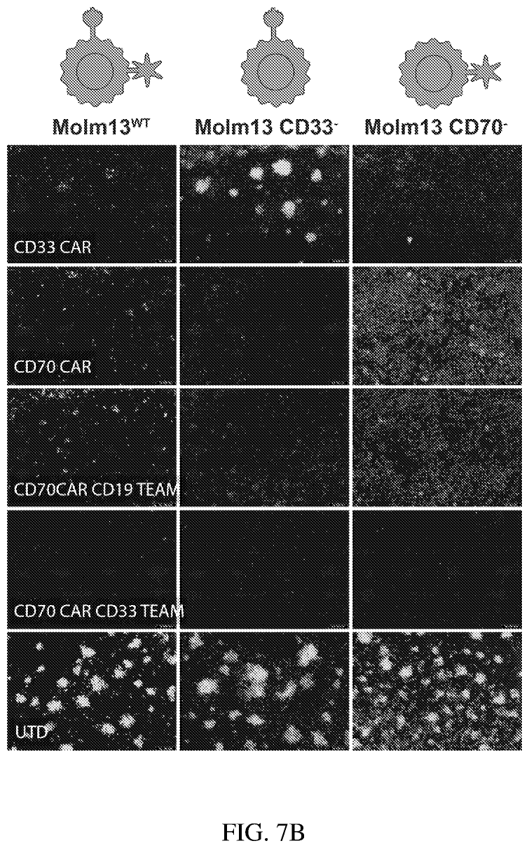

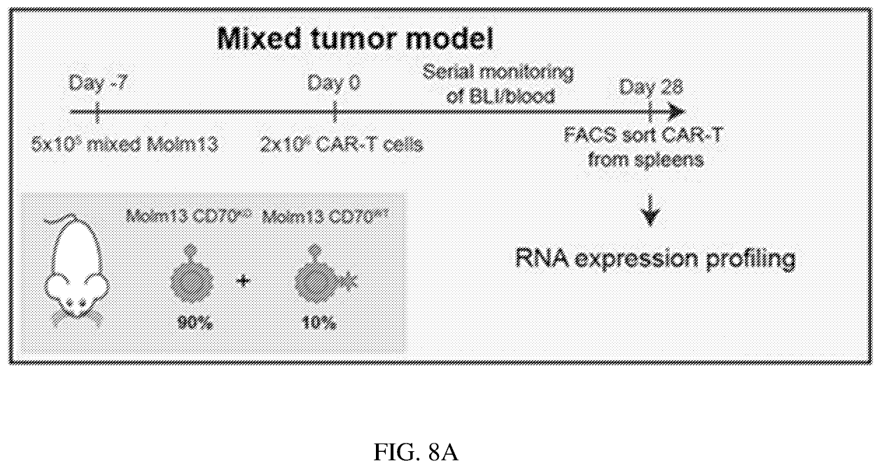

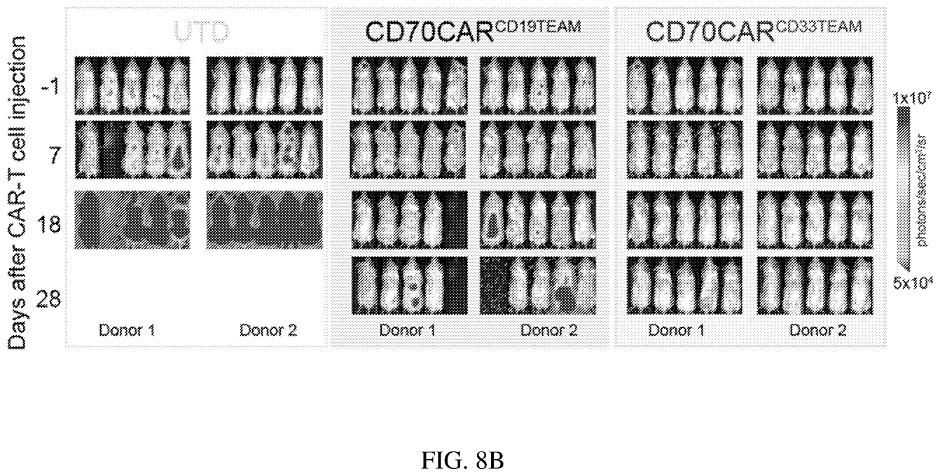

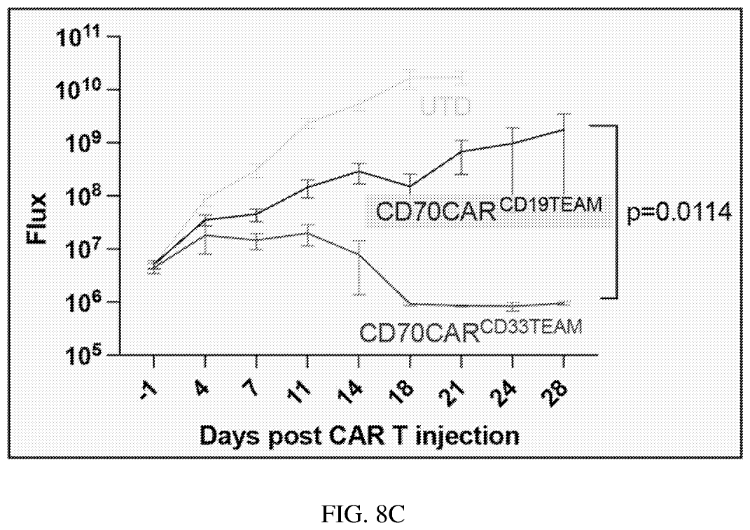

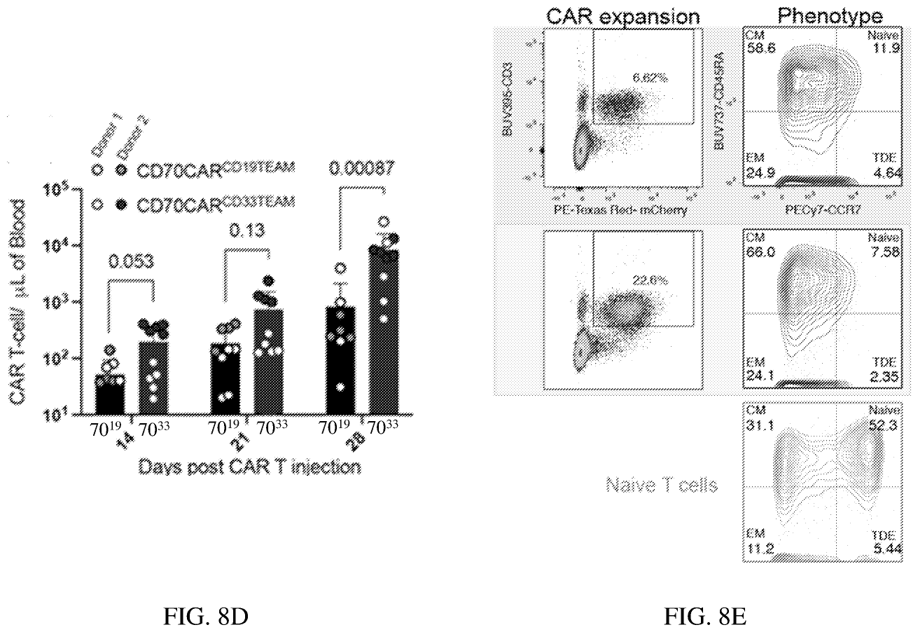

Next, CD70CAR^{CD33TEAM} was tested for effectiveness in xenograft models of AML in vivo. NSG mice were injected with 5×10⁵ mixed population Molm13 cells (90% Molm13^{CD70KO}, 10% Molm13^{WT}, n=10 mice/group) on day −7 which express the bioluminescent (BLI) reporter click beetle green (FIG. 8A). On day 0 mice received 2×10⁶ of either CD70CAR^{CD19TEAM} or CD70CAR^{CD33TEAM} cells (n=5 mice per individual T-cell donor per group). Serial BLI monitoring revealed tumor eradication in the CD70CAR^{CD33TEAM} group of mice, but uncontrolled tumor growth in the CD70CAR^{CD19TEAM} (FIGS. 8B-8C). Peripheral blood expansion was determined by flow cytometry days 14-28 after CAR-T injection (FIG. 8D). Peripheral blood CAR-T phenotypic profiling was also performed via flow cytometry from the blood of mice at day +21 after CAR-T injection compared to unstimulated and nontransduced, donor matched T-cells (FIG. 8E).

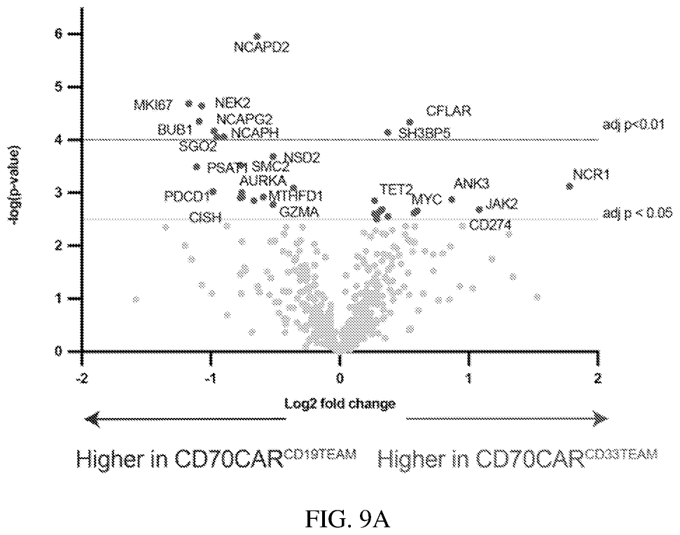

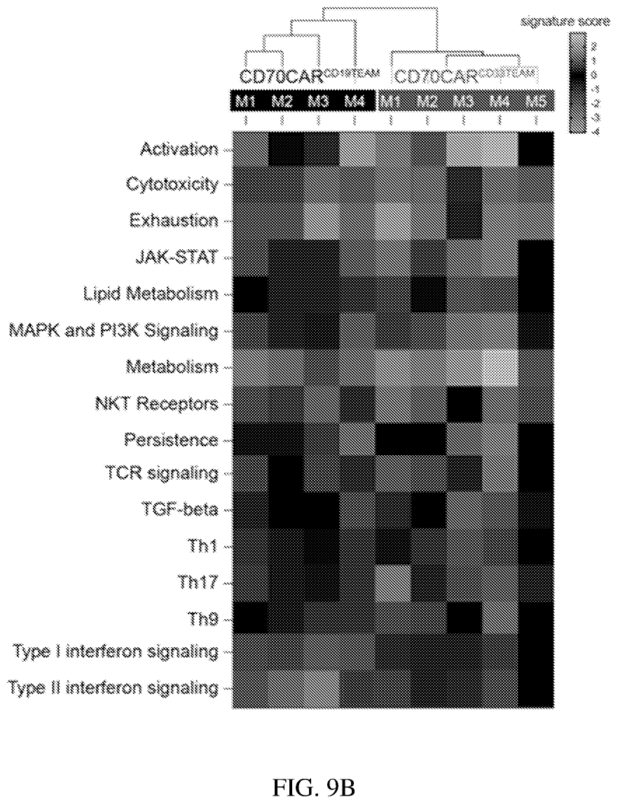

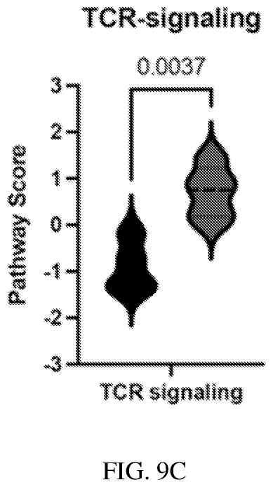

CD70CAR^{CD33TEAM} cells were also found to exhibit divergent expressional programs compared to CD70CAR^{CD19TEAM} cells in vivo. CAR-T cells were separated via fluorescence-activated cell sorting (FACS) from the spleens of the mice in the mixed tumor model. CAR-T cells were then lysed and underwent gene expression analysis via nanostring with the CAR-T characterization panel in addition to a custom ‘drop-in’ gene set (FIGS. 9A-9C).

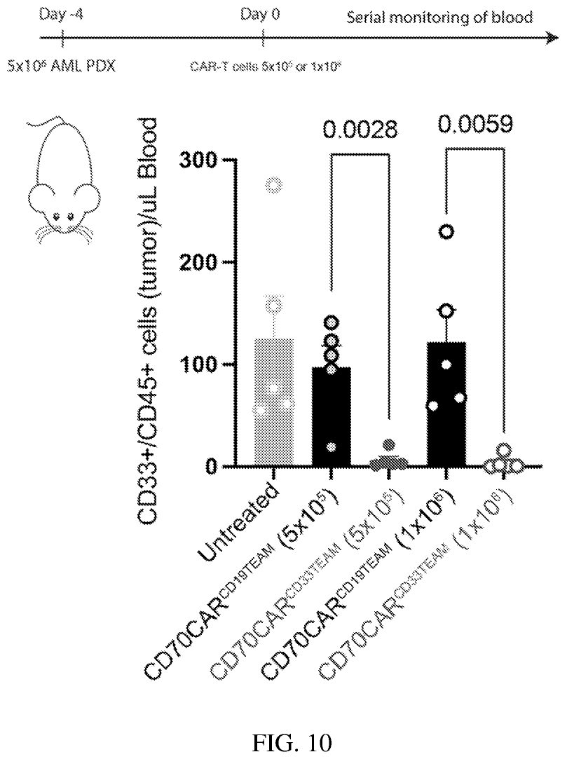

Next, it was determined if decreasing the dose of CAR-T cells administered to a patient derived xenograph AML mouse model altered treatment efficacy. A dose of 5×10⁵ and 1×10⁶ CD70 CAR CD33 TEAM CAR-T cells were administered (FIG. 10). Results showed a similar decrease in the number of CD33+/CD45+ cells (tumor) in this mice with both doses.

Overall, these findings demonstrate the potency of a CD70-targeted CAR which secretes a CD33-targeted T-cell engaging antibody molecule against tumor targets with variable CD33 and CD70 antigen expression.

One particularly well characterized AML antigen is CD33 which is expressed in up to 90% of leukemic blasts³¹, (also on normal myeloid cells and some progenitors but not CD34+ stem cells³²). Importantly, elimination of CD33+ cells via treatment of bone marrow autografts with a monoclonal antibody eliminates committed myeloid progenitors, however, while delayed, normal trilinear hematopoiesis occurs.³³ Unexpectedly, CD33 is also found on hepatocytes and treatment with the CD33 ADC, gemtuzumab ozogamicin (GO), has led to fatal liver toxicity in the form of veno-occlusive disease (VOD).34 CD33 can also be found on immunosuppressive MDSCs in the bone marrows of patients with AML. Recently, successful synergistic targeting of these MDSCs and AML using a CD3/CD33 bispecific T cell engager has been demonstrated in vitro.³⁵ Comprehensive AML surfaceome analysis of AML cell lines and primary patient samples projected that simultaneous targeting of CD33 and CD70 in AML would be feasible in over 97% of patients with non-overlapping bystander tissue toxicities.¹⁹

To simultaneously address antigenic heterogeneity, promote local anti-tumor activity through the recruitment of Tconventional, Treg and exhausted Teff, and eliminate immunosuppressive MDSCs, a CD3/CD33 TEAM secreting CD70 targeted CAR (CAR-TEAM) was generated and tested, based on a previously optimized CD70-targeted CAR-T platform.³⁶

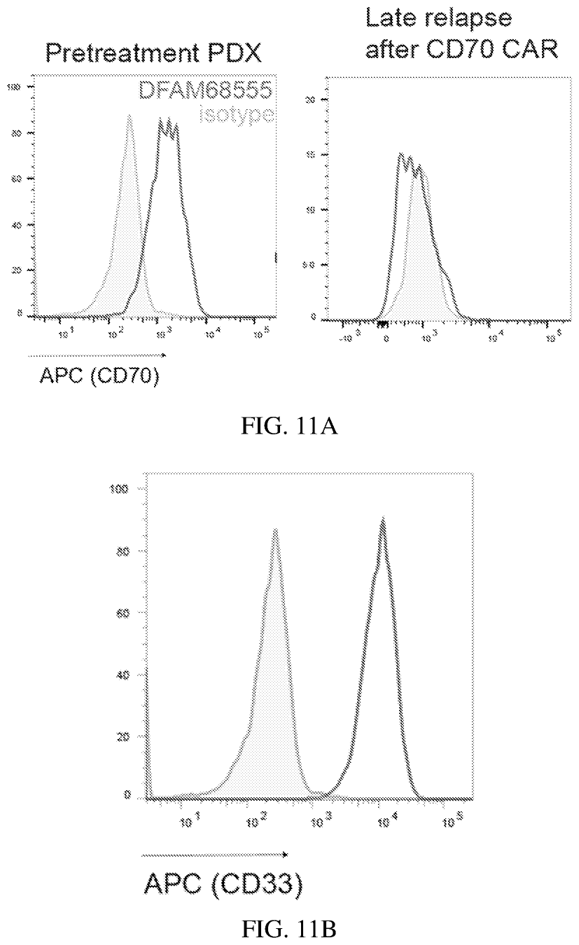

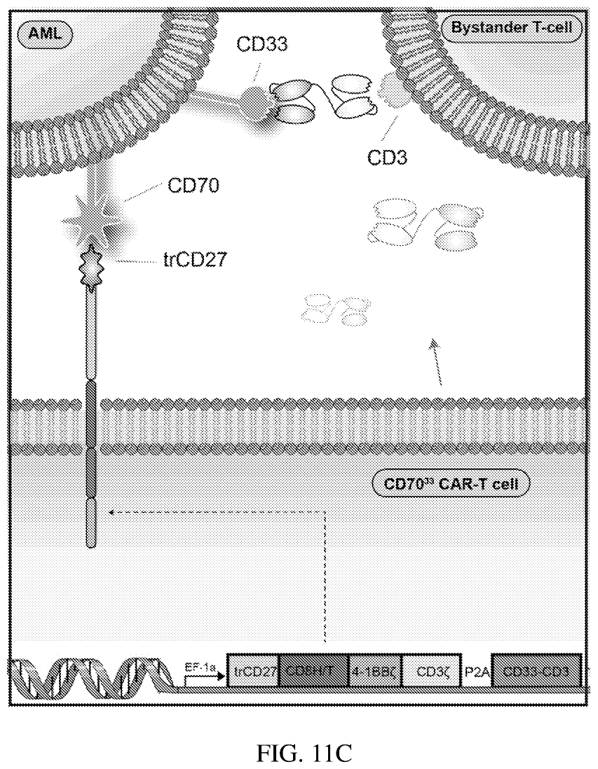

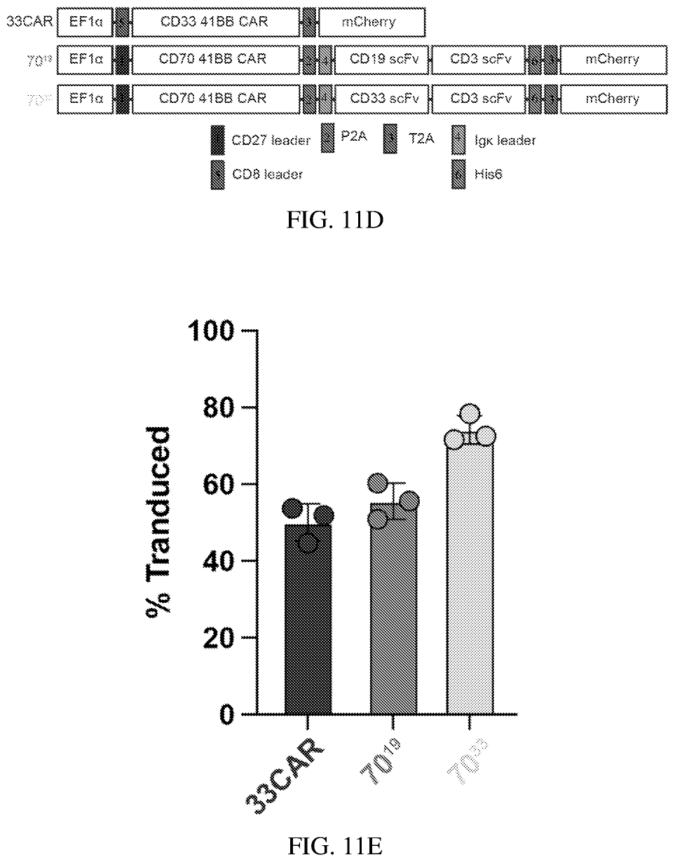

The superiority of a second generation ligand based CD70-targeting CAR construct with a modified hinge and transmembrane domain to abrogate protease-mediated CAR decapitation relative to an unmodified version in a patient-derived xenograft model of AML was previously demonstrated. (Leick et al. 2022) However, in some mice there was loss of CD70 antigen expression (FIG. 11A). CD33 is a highly expressed myeloid antigen on the majority of AML blasts (and, indeed, was expressed at high levels in the PDX model FIG. 11B) and it has been suggested that combinatorial targeting of CD33 and CD70 by CAR-T cells would eliminate the majority of AMLs with non-overlapping bystander off-tumor toxicities. (Perna et al. 2017) The optimized CD70-targeted CAR was therefore utilized as a platform to develop a dual targeting strategy by modifying it to secrete a CD33 targeted T cell engaging antibody molecule (TEAM) which was called 70³³ (FIG. 11C). For downstream testing this construct was compared to a CD33-targeted CAR (CAR33) and control version which secreted a CD19-targeting TEAM 70¹⁹, an antigen which is not found in typical AML cases (FIG. 11D). Despite the added payload of a secreted TEAM, transduction efficiencies were more than adequate across healthy donors' T cells and comparable to CAR33 (FIG. 11E).

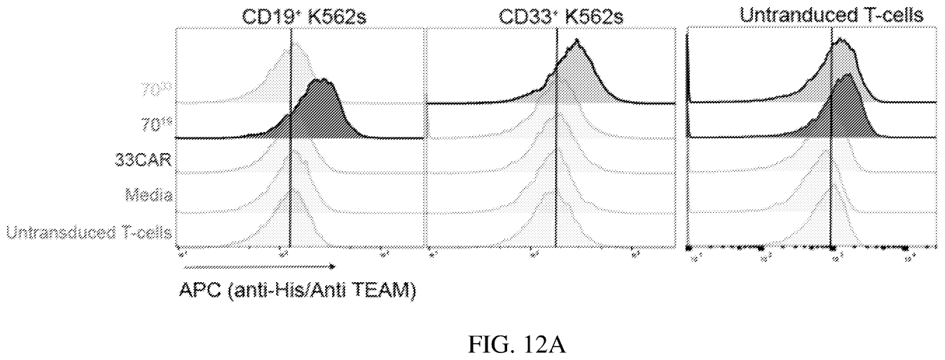

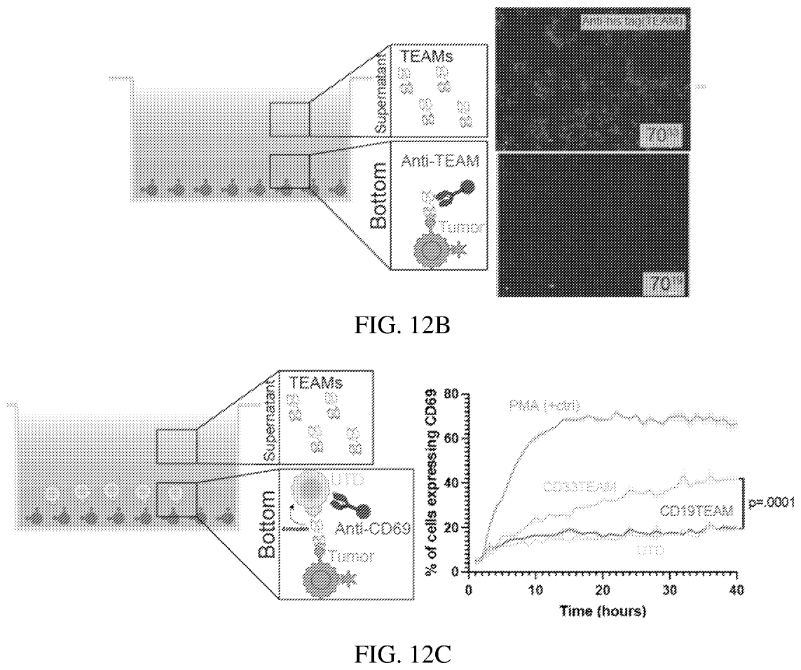

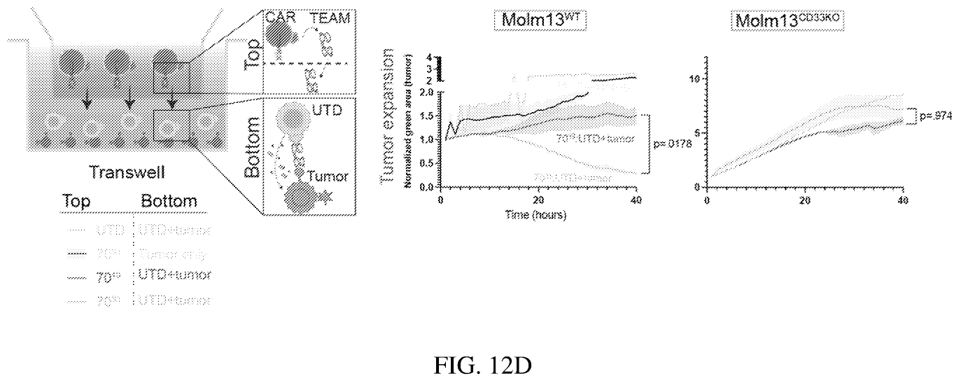

To ensure appropriate target binding of the TEAM molecules to their intended antigens supernatant was collected from jurkat T cells transduced with each of the constructs and incubated it with target cells expressing the relevant antigen and assayed for the presence of the TEAM via flow cytometry, (FIG. 12A). It was found that the 70³³ supernatant demonstrated binding to CD33 expressing K562s as well as untranduced T (UTD) cells, while the 70¹⁹ supernatant bound to CD19 expressing K562s and UTD. In a separate confirmatory assay 70³³ or 70¹⁹ supernatant was added to immobilized molm13 tumor targets followed by anti-His-PE antibody allowing for visualization of TEAM binding microscopically (FIG. 12B).

Next, to assess the ability of the TEAM molecules to activate bystander T-cells 70³³, 70¹⁹, or UTD supernatant were added to wells with immobilized tumor targets, UTD T cells, and antibody against the activation marker CD69. It was found that 70³³ supernatant led to significantly more UTD CD69 expression than 70¹⁹ (p.0001) which was indistinguishable from UTD supernatant (FIG. 12C). Finally, to ascertain the cytolytic capacity of the TEAM a transwell assay was performed in which TEAM producing cells were placed in the top of a transwell insert too small for cells to pass through but large enough for proteins to pass while tumor targets and UTDs were placed in bottom (FIG. 12D). Only when 70³³ producing cells were placed in the top of the transwell along with UTDs included on bottom was tumor eliminated (p.018). This phenomenon was target specific as Molm13^{CD33KO} grew at the same rate whether 70³³ or 70¹⁹ was in the top of the transwell insert (p.97).

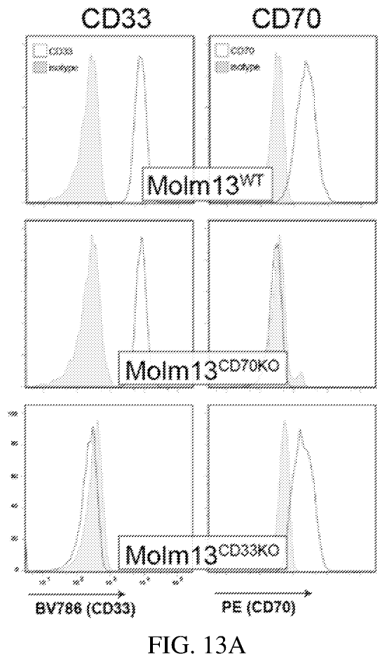

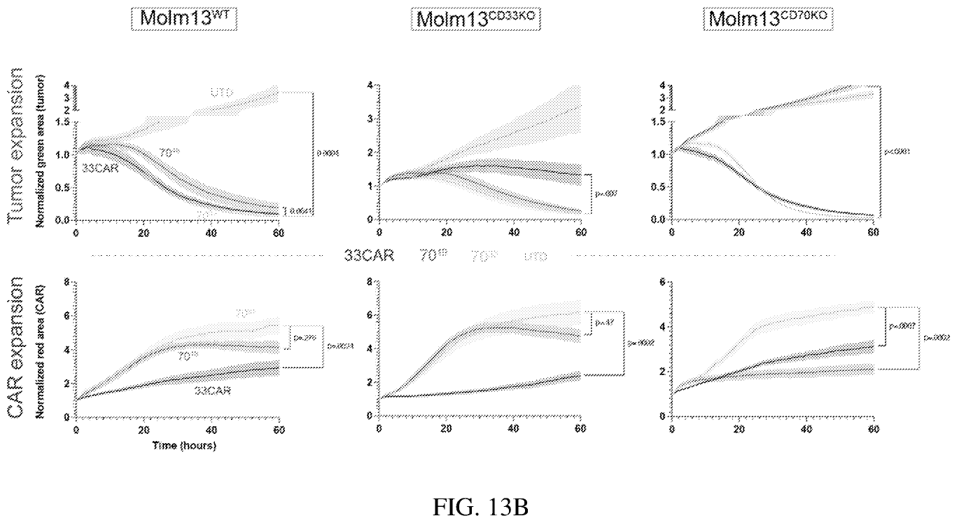

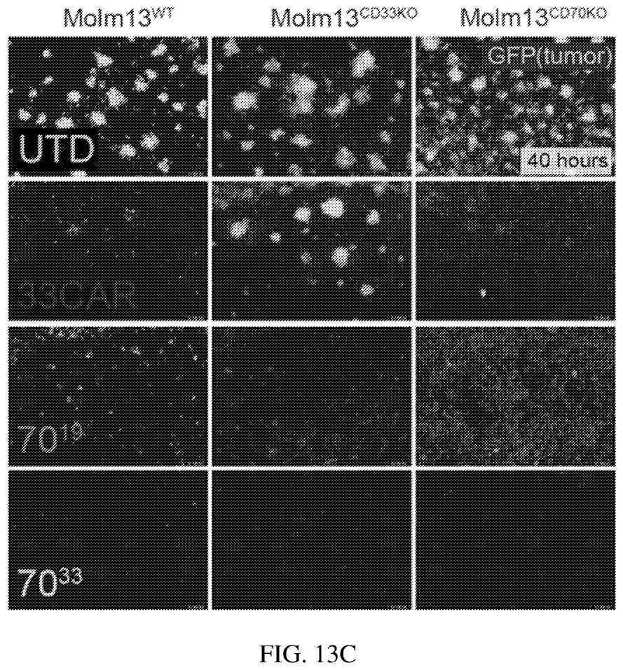

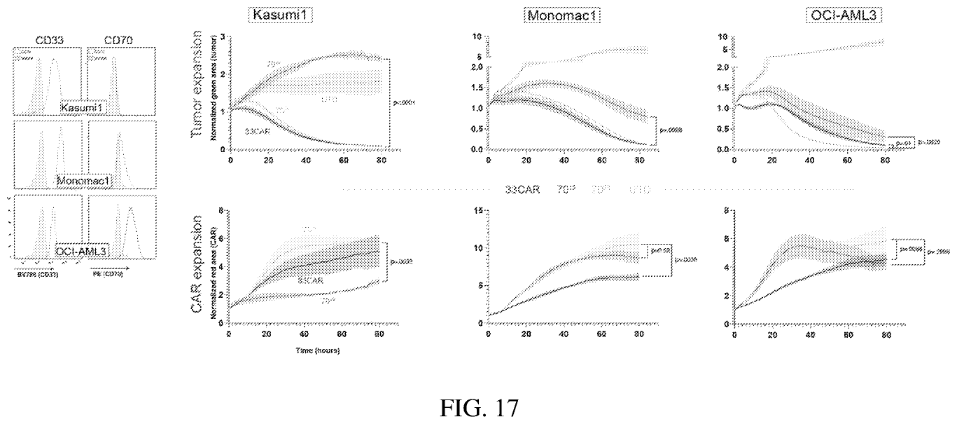

After confirming the specificity and cytolytic capacity of the CD33 TEAM molecule, how the entire construct performed under different levels of CD33 and CD70 antigen expression was investigated. The killing ability of CAR33, 70¹⁹, 70³³ was compared against wild type and engineered molm13 cell lines and it was found that 70³³ CAR-T cells mediated superior cytotoxicity against: molm13^{WT} relative to 70¹⁹ (p.064), molm13^{CD33KO} relative to CAR33 (p.007), and molm13^{CD70KO} relative to 70¹⁹ (p.0001) (FIGS. 13A-13C). 70³³ CAR-T cells also had the highest level of expansion across each of the permutations (FIG. 13B). Similar results were seen when 70³³ was used to target different native levels of CD33 and CD70 in the AML cell lines Kasumi1, Monomac1, and OCI-AML3 (FIG. 17).

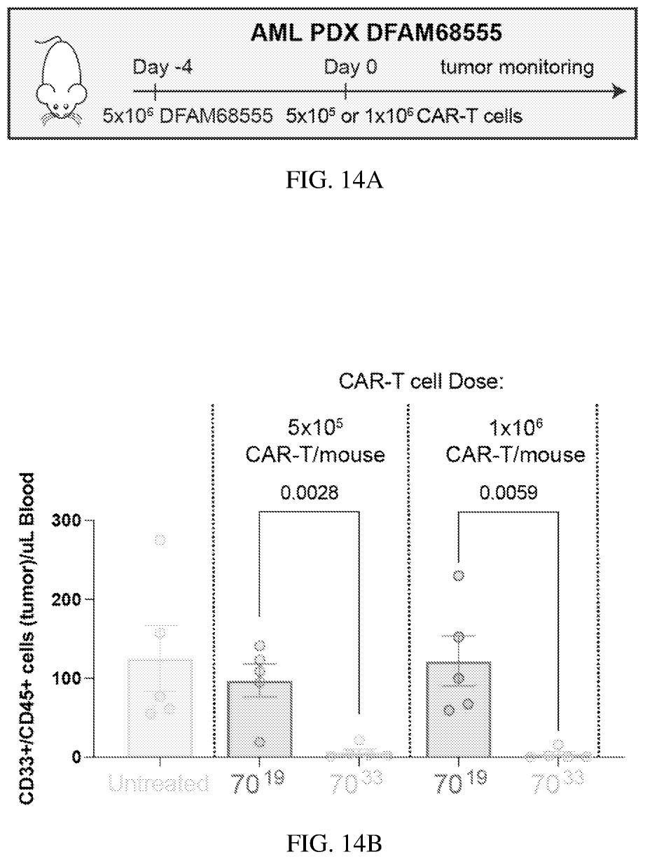

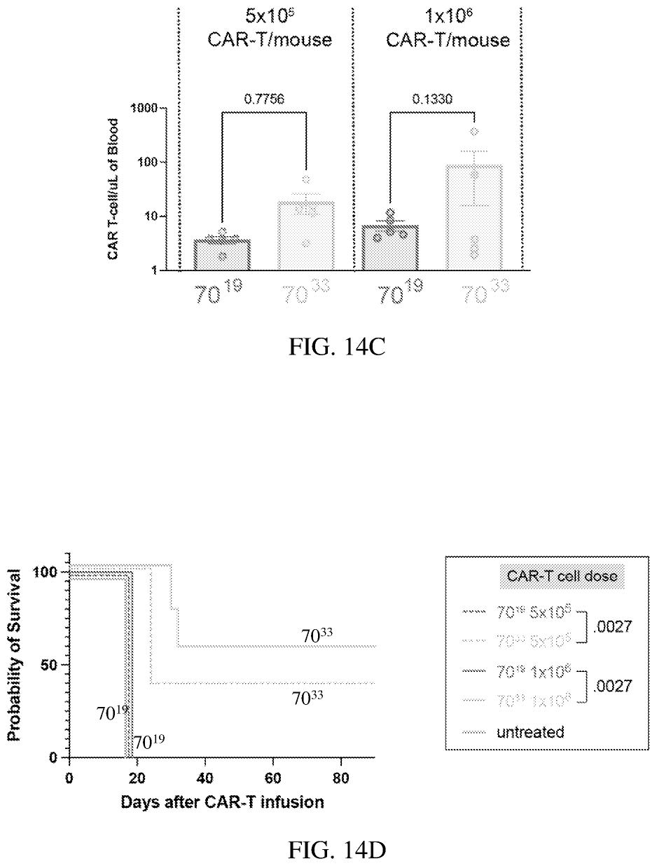

After establishing the in vitro potency of the dual targeting 70³³ system, the original PDX model was returned to, to ascertain if this strategy led to an improvement over the original optimized CD70 CAR. In a stress version of the PDX model NSG mice were injected with 5×10⁶ fresh PDX cells on day −4 followed by 5×10⁵ or 1×10⁶ 70³³ or 70¹⁹ CAR-T cells (½ and ¼ the previous treatment dose respectively) on day 0 (FIG. 14A). 70³³ CAR-T cells led to superior tumor control by day +14 (p.0028, p.0059 respectively, FIG. 14B), numerically higher expansion (p.78, p.13 respectively, FIG. 14C), and prolonged survival (p.027 for both, FIG. 14D) at both dose levels.

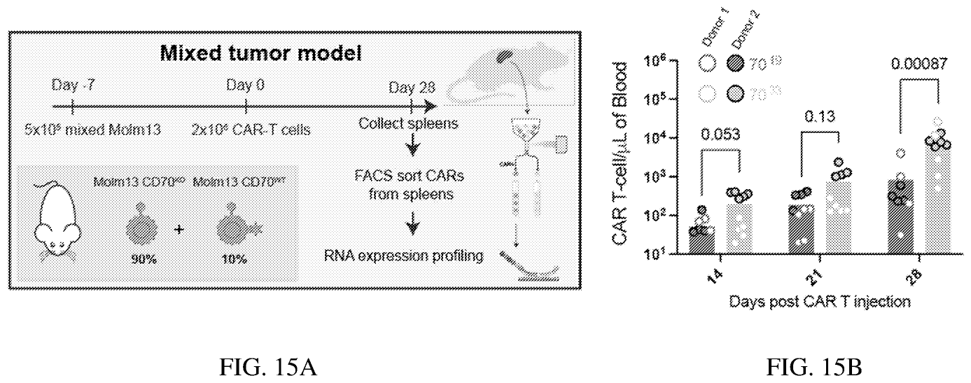

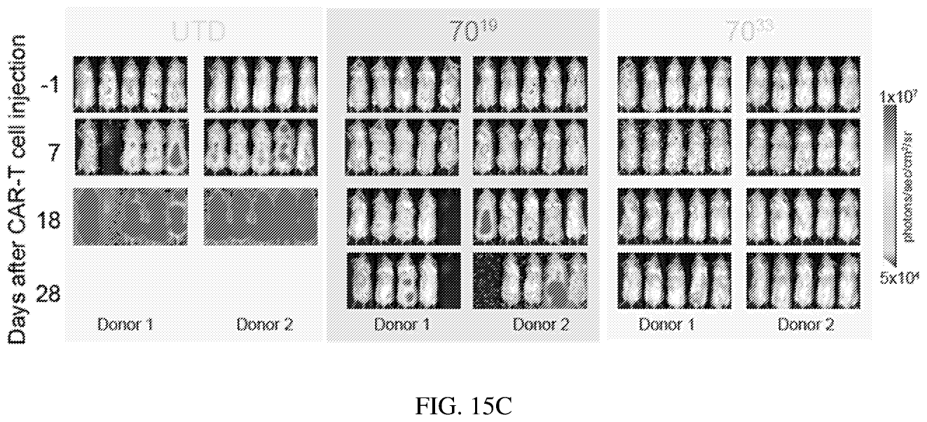

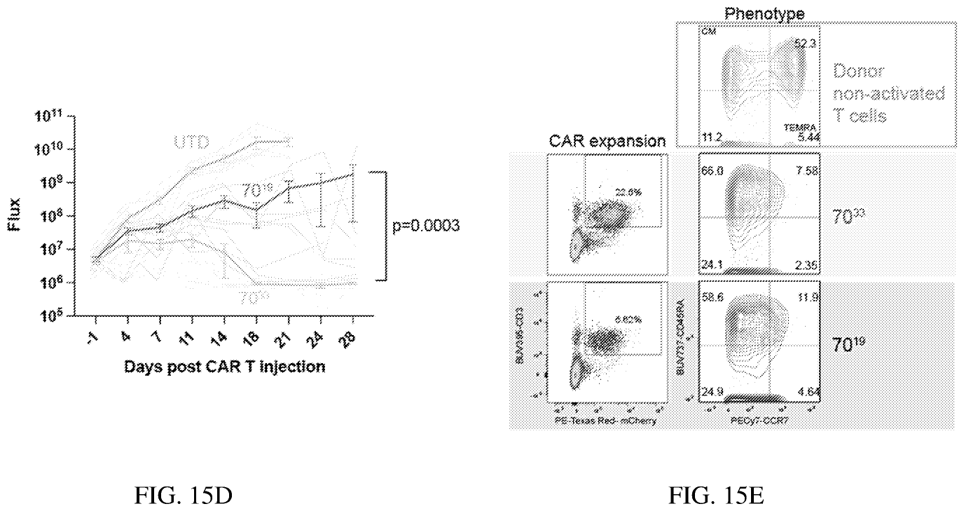

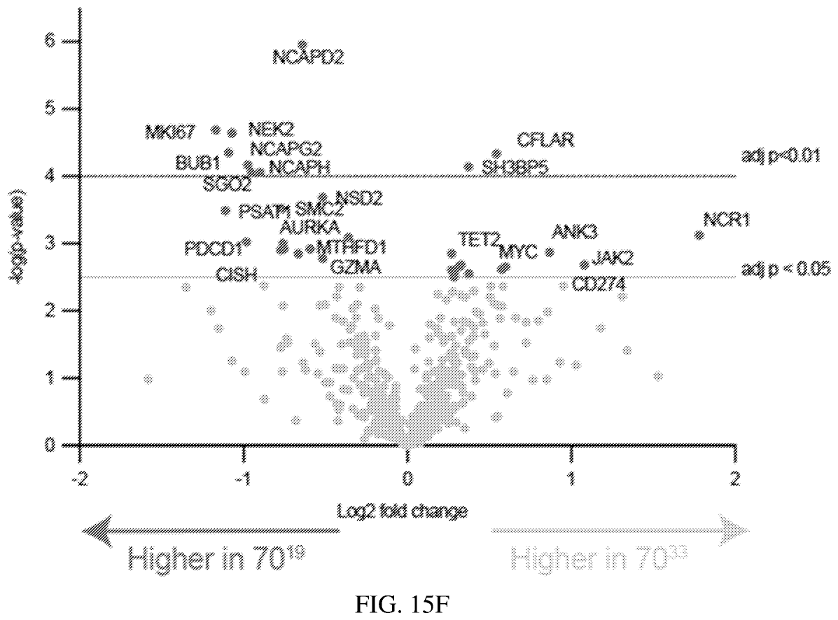

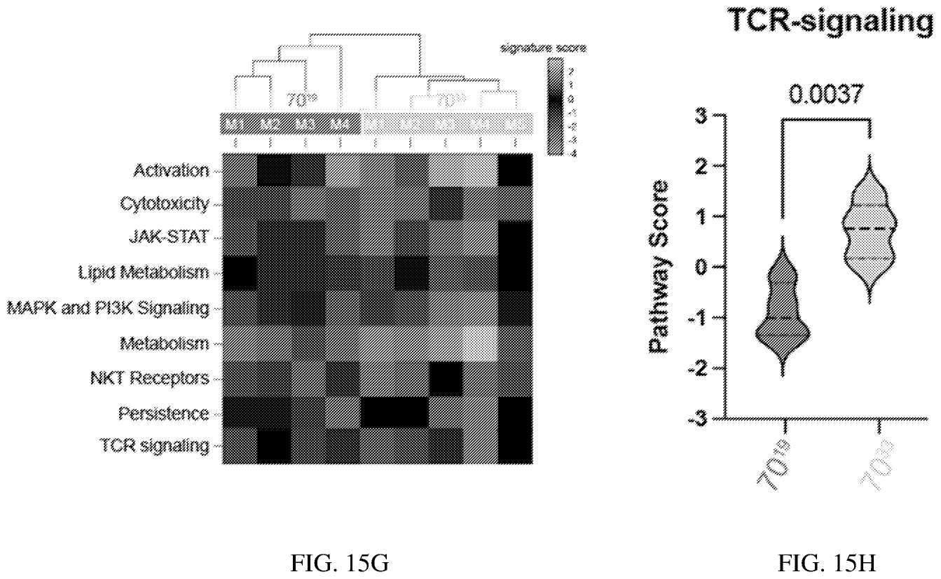

To more precisely model the CD70 antigen escape that had been previously identified after CD70 CAR monotherapy, a mixture of 5×10⁵ Molm13^{WT} and Molm13^{CD70KO} cells were injected into NSG mice on day −7 followed by 2×10⁶ 70¹⁹, 70³³, or UTD cells seven days later on day 0 (FIG. 15A). 70³³ CAR-T cells had significantly higher expansion relative to 70¹⁹ across time points (days +14; p=. 053,+21; p=0.13, +28; p=. 00087). Tumor as assessed by bioluminescent imaging was eradicated in 70³³ mice by day +18 by CAR-T cells from both healthy donors, while 70¹⁹ CAR-T cells failed to control tumor growth (p=0.0003, FIGS. 15C-15D). At day +21 both constructs adopted primarily a central-memory like phenotype (FIG. 15E).

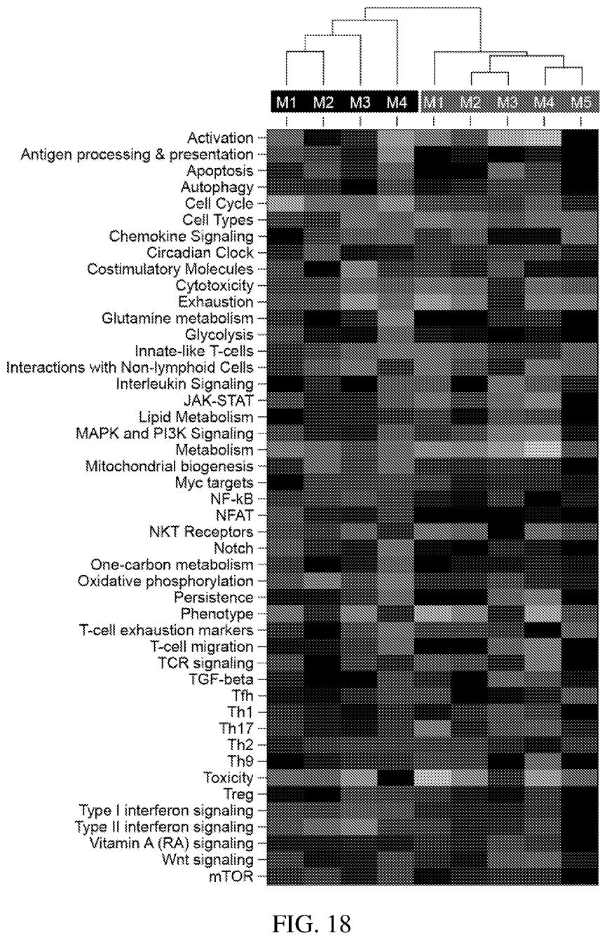

To understand if there were any divergent transcriptional programs driving these two CAR-T cells' behavior, the spleens of mice from each group at day +28 were collected and FACS sorting was performed for CAR-T cells which then underwent targeted gene expression analysis (FIG. 15F). Key players in CAR-T cell proliferative signaling like JAK2 and MYC were higher in 70³³ while negative regulators of T cell function like CISH and PDCD1 (PD-1) were higher in 70¹⁹ CAR-T cells. Interestingly, as adoption of an NK-cell like phenotype has been associated with CAR-T dysfunction (Good et al. 2021), the gene with the highest fold-change in expression in 70³³ relative to 70¹⁹ CAR-T cells was the NK-cell receptor NCR1. Unbiased pathway clustering resulted in lumping of the respective CAR constructs with key differences in signature scores in activation, cytotoxicity, and persistence (FIG. 15G, FIG. 18). A significantly higher pathway score for TCR signaling in the 70³³ CAR-T cells is suggestive of simultaneous CAR-T activation through the CAR as well as through the TCR (via engagement with TEAMs) (FIG. 15H).

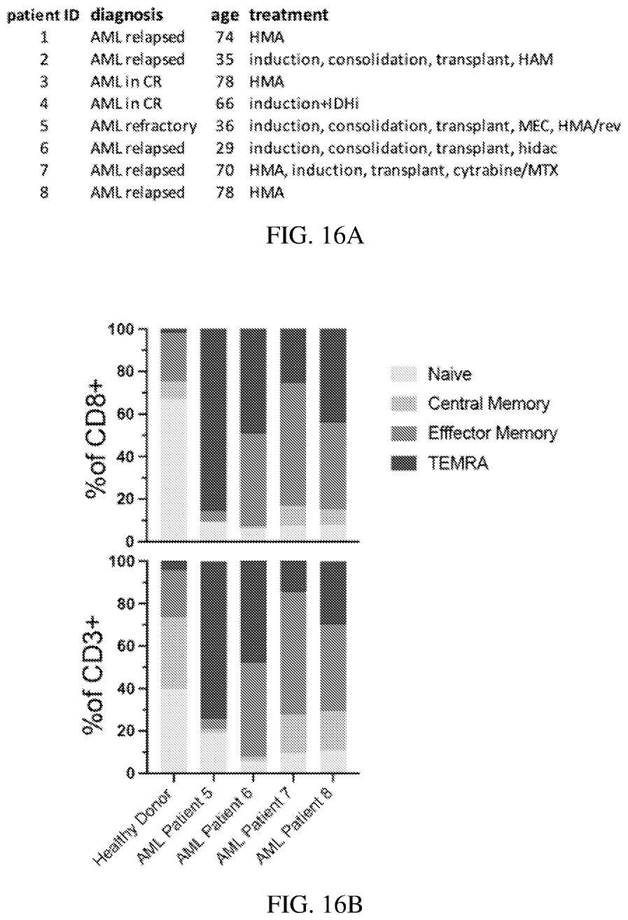

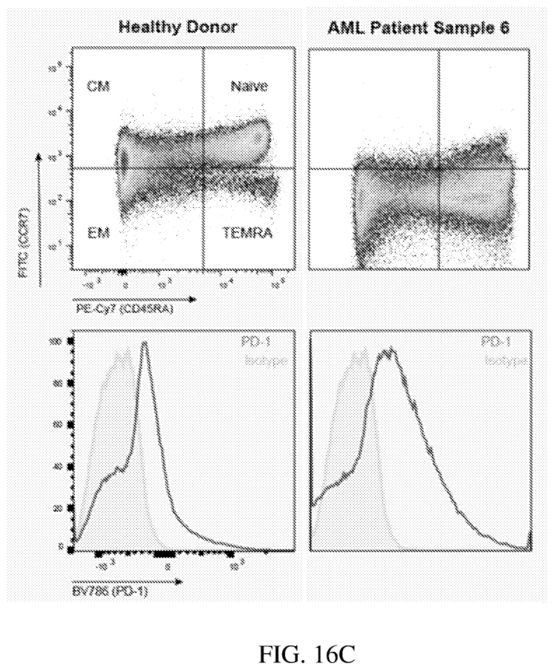

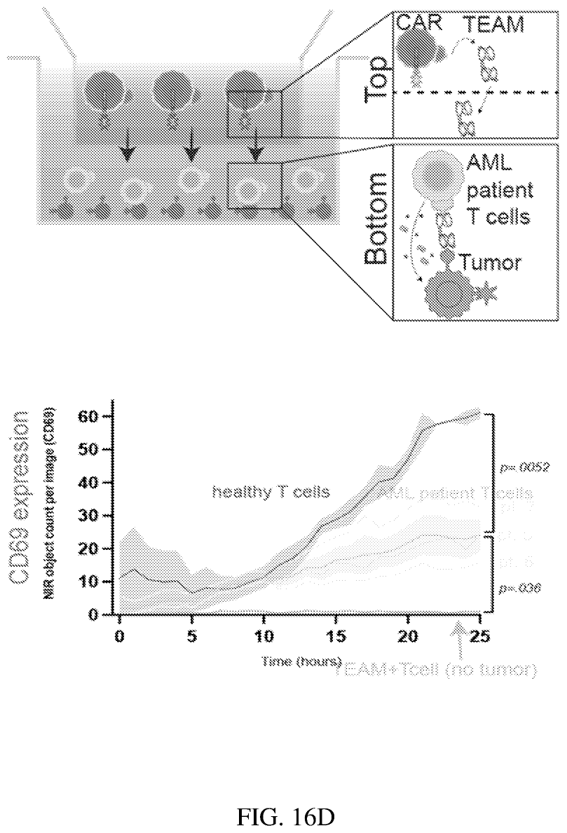

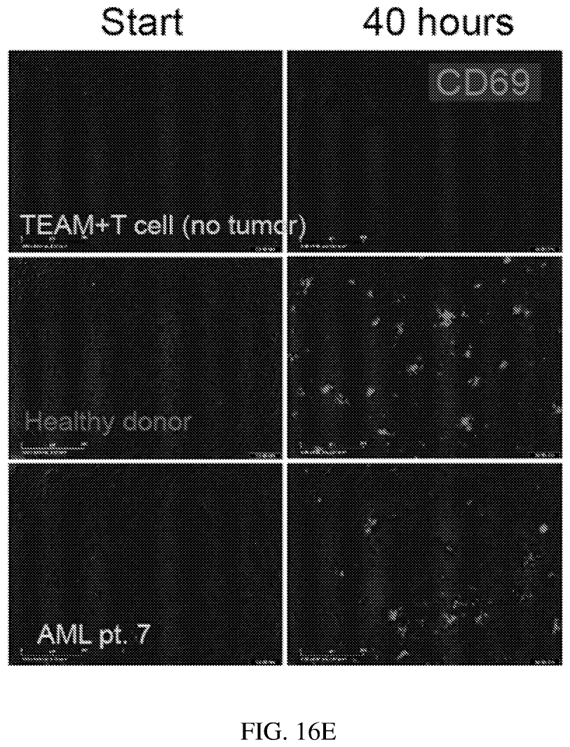

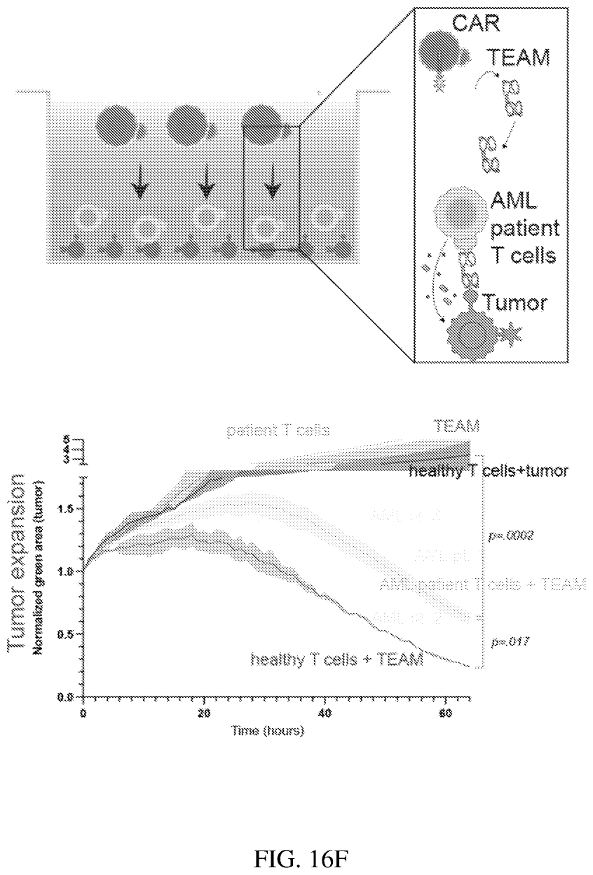

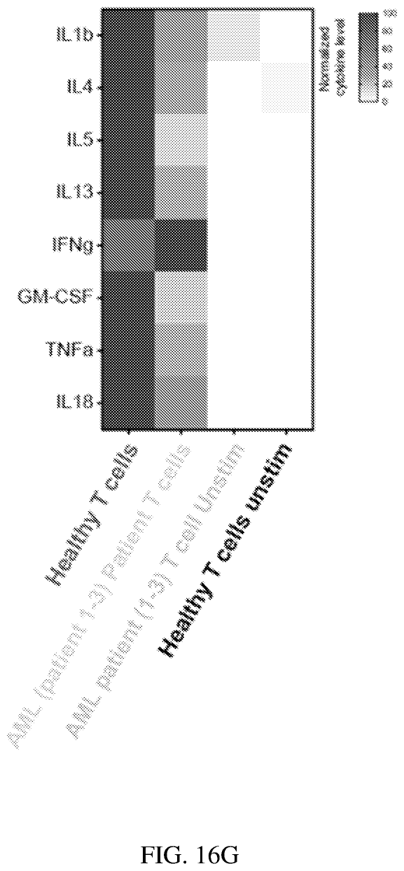

One limitation of CAR-T cell therapy in cancer patients has been intrinsically defective T cells used as manufacturing substrate due to immunosuppressive effects of the cancer itself, or T cell damage due to intensive chemoimmunotherapy. (Das et al. 2019; Fraietta et al. 2018) To assess the ability of the TEAM described hereinto successfully leverage AML patients' T cells to target AML, PBMC was obtained from AML patients who had undergone a variety of intensive chemo and immunotherapies including allogeneic bone marrow transplantation (FIG. 16A). T cells were isolated and flow cytometric phenotyping was performed. It was found that AML patients' T cells had a more differentiated and exhausted phenotype compared to healthy donor T cells (FIG. 16B-16C). AML patient cells demonstrated significant activation in the presence of tumor (p.036) though at reduced levels compared to healthy donor T cells (p.0052, FIG. 16G). To assess the cytotoxicity of AML patients' T cells acting as redirected bystanders via the TEAM, their isolated T cells were cocultured with TEAM producer cells and AML targets. AML patients uniformly demonstrated significant cytotoxicity (p.0002, though were less potent than healthy donor T cells p.017, FIG. 16D). T cells from AML patients produced inflammatory cytokines, though at lower numbers than healthy donor T cells, intriguingly except for IFNg (FIG. 16E).

### REFERENCES

1. Dohner H, Weisdorf D J, Bloomfield C D. Acute Myeloid Leukemia. New England Journal of Medicine 2015; 373 (12): 1136-1152. DOI: 10.1056/NEJMra1406184.
2. Guerra V A, DiNardo C, Konopleva M. Venetoclax-based therapies for acute myeloid leukemia. Best practice & research Clinical haematology 2019; 32 (2): 145-153. (In eng). DOI: 10.1016/j.beha.2019.05.008.
3. Yarchoan M, Hopkins A, Jaffee E M. Tumor Mutational Burden and Response Rate to PD-1 Inhibition. The New England journal of medicine 2017; 377 (25): 2500-2501. (In eng). DOI: 10.1056/NEJMc1713444.
4. Daver N G, Basu S, Garcia-Manero G, et al. Phase IB/II study of nivolumab with azacytidine (AZA) in patients (pts) with relapsed AML. Journal of Clinical Oncology 2017; 35 (15_suppl): 7026-7026. DOI: 10.1200/JCO.2017.35.15_suppl.7026.
5. Lawrence M S, Stojanov P, Polak P, et al. Mutational heterogeneity in cancer and the search for new cancer-associated genes. Nature 2013; 499 (7457): 214-218. (In eng). DOI: 10.1038/nature12213.
6. Papaemmanuil E, Gerstung M, Bullinger L, et al. Genomic Classification and Prognosis in Acute Myeloid Leukemia. New England Journal of Medicine 2016; 374 (23): 2209-2221. DOI: 10.1056/NEJMoa1516192.
7. Pyzer A R, Stroopinsky D, Rosenblatt J, et al. Myeloid-Derived Suppressor Cells Are Expanded in Patients with AML and Are Dependent on MUC1 Expression. Blood 2014; 124 (21): 226-226. DOI: 10.1182/blood. V124.21.226.226.
8. Alex A A, Tartour E, Gey A, et al. Myeloid Derived Suppressor Cells in Acute Leukemia and Its Association with Conventional Cytogenetic and Molecular Risk Factors. Blood 2012; 120 (21): 1446-1446. DOI: 10.1182/blood. V120.21.1446.1446.
9. Williams P, Basu S, Garcia-Manero G, et al. The distribution of T-cell subsets and the expression of immune checkpoint receptors and ligands in patients with newly diagnosed and relapsed acute myeloid leukemia. Cancer 2019; 125 (9): 1470-1481. DOI: 10.1002/cncr.31896.
10. Schuster S J, Svoboda J, Chong E A, et al. Chimeric Antigen Receptor T Cells in Refractory B-Cell Lymphomas. New England Journal of Medicine 2017; 377 (26): 2545-2554. DOI: 10.1056/NEJMoa1708566.
11. Neelapu S S, Locke F L, Bartlett N L, et al. Axicabtagene Ciloleucel CAR T-Cell Therapy in Refractory Large B-Cell Lymphoma. New England Journal of Medicine 2017; 377 (26): 2531-2544. DOI: 10.1056/NEJMoa1707447.
12. Mardiros A, Dos Santos C, McDonald T, et al. T cells expressing CD123-specific chimeric antigen receptors exhibit specific cytolytic effector functions and antitumor effects against human acute myeloid leukemia. Blood 2013; 122 (18): 3138-3148. DOI: 10.1182/blood-2012-12-474056.
13. Luo Y, Chang L-J, Hu Y, Dong L, Wei G, Huang H. First-in-Man CD123-Specific Chimeric Antigen Receptor-Modified T Cells for the Treatment of Refractory Acute Myeloid Leukemia. Blood 2015; 126 (23): 3778-3778. DOI: 10.1182/blood. V126.23.3778.3778.
14. Wang Q-s, Wang Y, Lv H-y, et al. Treatment of CD33-directed Chimeric Antigen Receptor-modified T Cells in One Patient With Relapsed and Refractory Acute Myeloid Leukemia. Molecular Therapy 2015; 23 (1): 184-191. DOI: doi.org/10.1038/mt.2014.164.
15. Kim M Y, Yu K-R, Kenderian S S, et al. Genetic Inactivation of CD33 in Hematopoietic Stem Cells to Enable CAR T Cell Immunotherapy for Acute Myeloid Leukemia. Cell 2018; 173 (6): 1439-1453.e19. DOI: doi.org/10.1016/j.cell.2018.05.013.
16. Jetani H, Garcia-Cadenas I, Nerreter T, et al. CAR T-cells targeting FLT3 have potent activity against FLT3−ITD+ AML and act synergistically with the FLT3-inhibitor crenolanib. Leukemia 2018; 32 (5): 1168-1179. DOI: 10.1038/s41375-018-0009-0.
17. Hofmann S, Schubert M L, Wang L, et al. Chimeric Antigen Receptor (CAR) T Cell Therapy in Acute Myeloid Leukemia (AML). Journal of clinical medicine 2019; 8 (2) (In eng). DOI: 10.3390/jcm8020200.
18. Melao A. FDA Suspends UCART123 Trials After Patient Death. Immuno-oncology News. Sep. 7, 2017.
19. Perna F, Berman S H, Soni R K, et al. Integrating Proteomics and Transcriptomics for Systematic Combinatorial Chimeric Antigen Receptor Therapy of AML. Cancer Cell 2017; 32 (4): 506-519.e5. DOI: doi.org/10.1016/j.ccell.2017.09.004.
20. Riether C, Schürch CM, Bührer E D, et al. CD70/CD27 signaling promotes blast stemness and is a viable therapeutic target in acute myeloid leukemia. J Exp Med 2017; 214 (2): 359-380. (In eng). DOI: 10.1084/jem.20152008.
21. Ochsenbein A. Argx-110 Targeting CD70, in Combination with Azacitidine, Shows Favorable Safety
Profile and Promising Anti-Leukemia Activity in Newly Diagnosed AML Patients in an Ongoing Phase 1/2 Clinical Trial. Abstract 2680. American Society of Hematology Annual Meeting. San Diego, CA2018.
22. Slaney C Y, Wang P, Darcy P K, Kershaw M H. CARs versus BiTEs: A Comparison between T Cell-Redirection Strategies for Cancer Treatment. Cancer Discov 2018; 8 (8): 924-934. (In eng). DOI: 10.1158/2159-8290.Cd-18-0297.
23. Majzner R G, Mackall C L. Clinical lessons learned from the first leg of the CAR T cell journey. Nature Medicine 2019; 25 (9): 1341-1355. DOI: 10.1038/s41591-019-0564-6.
24. Fry T J, Shah N N, Orentas R J, et al. CD22-targeted CAR T cells induce remission in B-ALL that is naive or resistant to CD19-targeted CAR immunotherapy. Nat Med 2018; 24 (1): 20-28. (In eng). DOI: 10.1038/nm.4441.
25. Shah N N, Zhu F, Schneider D, et al. Results of a phase I study of bispecific anti-CD19, anti-CD20 chimeric antigen receptor (CAR) modified T cells for relapsed, refractory, non-Hodgkin lymphoma. Journal of Clinical Oncology 2019; 37 (15_suppl): 2510-2510. DOI: 10.1200/JCO.2019.37.15_suppl.2510.
26. Gardner R, Annesley C, Finney O, et al. Early Clinical Experience of CD19×CD22 Dual Specific CAR T Cells for Enhanced Anti-Leukemic Targeting of Acute Lymphoblastic Leukemia. Blood 2018; 132:278-278. DOI: 10.1182/blood-2018-99-113126.
27. Amrolia P J, Wynn R, Hough R, et al. Simultaneous Targeting of CD19 and CD22: Phase I Study of AUTO3, a Bicistronic Chimeric Antigen Receptor (CAR) T-Cell Therapy, in Pediatric Patients with Relapsed/Refractory B-Cell Acute Lymphoblastic Leukemia (r/r B-ALL): Amelia Study. Blood 2018; 132 (Supplement 1): 279-279. DOI: 10.1182/blood-2018-99-118616.
28. Hossain N, Sahaf B, Abramian M, et al. Phase I Experience with a Bi-Specific CAR Targeting CD19 and CD22 in Adults with B-Cell Malignancies. Blood 2018; 132:490-490. DOI: 10.1182/blood-2018-99-110142.
29. Schultz L, Davis K, Baggott C, et al. Phase 1 Study of CD19/CD22 Bispecific Chimeric Antigen Receptor (CAR) Therapy in Children and Young Adults with B Cell Acute Lymphoblastic Leukemia (ALL). Blood 2018; 132:898-898. DOI: 10.1182/blood-2018-99-117445.
30. Choi B D, Yu X, Castano A P, et al. CAR-T cells secreting BiTEs circumvent antigen escape without detectable toxicity. Nature Biotechnology 2019; 37 (9): 1049-1058. DOI: 10.1038/s41587-019-0192-1.
31. Hauswirth A W, Florian S, Printz D, et al. Expression of the target receptor CD33 in CD34+/CD38−/CD123+ AML stem cells. European Journal of Clinical Investigation 2007; 37 (1): 73-82. DOI: 10.1111/j.1365-2362.2007.01746.x.
32. Nguyen D H, Ball E D, Varki A. Myeloid precursors and acute myeloid leukemia cells express multiple CD33-related Siglecs. Experimental Hematology 2006; 34 (6): 728-735. DOI: doi.org/10.1016/j.exphem.2006.03.003.
33. Robertson M J, Soiffer R J, Freedman A S, et al. Human bone marrow depleted of CD33-positive cells mediates delayed but durable reconstitution of hematopoiesis: clinical trial of MY9 monoclonal antibody-purged autografts for the treatment of acute myeloid leukemia. Blood 1992; 79 (9): 2229-36. (In eng).
34. Rajvanshi P, Shulman H M, Sievers E L, McDonald G B. Hepatic sinusoidal obstruction after gemtuzumab ozogamicin (Mylotarg) therapy. Blood 2002; 99 (7): 2310-4. (In eng). DOI: 10.1182/blood.v99.7.2310.
35. Jitschin, R. et al. CD33/CD3-bispecific T-cell engaging (BiTE®) antibody construct targets monocytic AML myeloid-derived suppressor cells. Journal for ImmunoTherapy of Cancer vol. 6 Preprint at https://doi.org/10.1186/s40425-018-0432-9 (2018).
36. Leick, M. B. et al. Non-cleavable hinge enhances avidity and expansion of CAR-T cells for acute myeloid leukemia. Cancer Cell 40, 494-508.e5 (2022).

1. A cell comprising a chimeric antigen receptor (CAR) that binds to CD70 and a T-cell engaging antibody molecule (TEAM) that binds to CD33.

2. The cell of claim 1, wherein the cell is an immune cell.

3. The cell of claim 2, wherein the immune cell is a T-cell, a NK cell, a dendritic cell, a macrophage, a B cell, a neutrophil, an eosinophil, a basophil, a mast cell, a myeloid derived suppressor cell, a mesenchymal stem cell, a precursor thereof, or a combination.

4. The cell of claim 3, wherein the immune cell is a T-cell.

5. The cell of any one of claims 1-4, wherein the cell is collected from a subject, optionally a human subject.

6. The cell of any one of claims 1-5, wherein the CAR comprises:
(i) an extracellular target binding domain comprising a polypeptide that binds CD70;
(ii) a transmembrane domain; and
(iii) an intracellular signaling domain.

7. The cell of claim 6, wherein the extracellular target binding domain comprises the CD70-binding domain of CD27.

8. The cell of claim 7, wherein extracellular target binding domain comprises the extracellular domain of CD27.

9. The cell of claim 8, wherein the extracellular target binding domain comprises an amino acid sequence that is at least 80% identical to the amino acid sequence of any one of SEQ ID NOs: 1, 8, or 9.

10. The cell of claim 9, wherein the extracellular target binding domain comprises the amino acid sequence of any one of SEQ ID NOs: 1, 8, or 9.

11. The cell of claim 6, wherein the extracellular target binding domain comprises an anti-CD70 antibody, optionally an scFv.

12. The cell of any one of claims 6-11, wherein the transmembrane domain is the transmembrane domain of CD27.

13. The cell of any one of claims 6-12, wherein the intracellular signaling domain comprises (i) an ITAM-containing signaling domains and/or (ii) one or more signaling domains from one or more co-stimulatory proteins or cytokine receptors.

14. The cell of claim 13, wherein the intracellular signaling domain comprises a CD3γ, CD3ε, CD3δ, or CD3ζ domain.

15. The cell of claim 14, wherein the intracellular signaling domain comprises a CD3ζ domain.

16. The cell of any one of claims 6-15, wherein the costimulatory domain comprises a CD28, 4-1BB, 2B4, KIR, OX40, ICOS, MYD88, IL2 receptor, or SynNotch domain.

17. The cell of claim 16, wherein the costimulatory domain comprises a 4-1BB domain.

18. The cell of any one of claims 1-17, wherein the extracellular target binding domain further comprises a signal peptide, optionally wherein the signal peptide comprises a CD27 signal peptide.

19. The cell of any one of claims 1-18, wherein the CAR comprises an amino acid sequence that is at least 80% identical to the amino acid sequence of any one of SEQ ID NOs: 2-7.

20. The cell of any one of claims 1-19, wherein the CAR comprises the amino acid sequence of any one of SEQ ID NO: 2-7.

21. The cell of any one of claims 1-20, wherein the TEAM comprises an anti-CD33 antibody or a functional fragment thereof.

22. The cell of claim 21, wherein the anti-CD33 antibody is selected from the group consisting of a fragment antigen-binding region (Fab region), a single-chain variable fragment (scFv), a diabody, a nanobody or a monoclonal antibody.

23. The cell of claim 21 or claim 22, wherein the anti-CD33 antibody is an scFv.

24. The cell of any one of claims 21-23, wherein the anti-CD33 antibody comprises a VH domain having the amino acid sequence of SEQ ID NO: 20 and/or a VL domain having the amino acid sequence of SEQ ID NO: 19.

25. The cell of claim 24, wherein the VH domain is N-terminal of the VL domain.

26. The cell of claim 24, wherein the VL domain is N-terminal of the VH domain.

27. The cell of any one of claims 1-26, wherein the TEAM comprises an immune cell binding moiety, optionally the immune cell binding moiety binds CD3, CD8, CD4, CXCR3, CCR4, GARP, LAP, CD25, CTLA-4, or CD16.

28. The cell of claim 27, wherein the immune cell binding moiety is selected from the group consisting of a fragment antigen-binding region (Fab region), a single-chain variable fragment (scFv), a diabody, a nanobody or a monoclonal antibody.

29. The cell of claim 27 or claim 28, wherein the immune cell binding moiety is an anti-CD3 scFv.

30. The cell of any one of claims 27-29, wherein the TEAM comprises a linker between the anti-CD33 antibody and the immune cell binding moiety.

31. The cell of claim 30, wherein the linker is a non-cleavable linker, optionally a (GGGGS) 3 (SEQ ID NO: 27) linker.

32. The cell of any one of claims 1-31, wherein the TEAM further comprises a secretion tag, optionally a IgK secretion tag.

33. The cell of any one of claims 1-32, wherein the TEAM comprises an amino acid sequence that is at least 85% identical to any one of SEQ ID NOs: 17-18.

34. The cell of any one of claims 1-33, wherein the cell comprises a polynucleotide molecule comprising a nucleic acid sequence encoding an amino acid sequence of any one of SEQ ID NOs: 17-18.

35. The cell of any one of claims 1-34, wherein the nucleic acid sequence encoding the TEAM is codon-optimized.

36. The cell of any one of claims 1-35, comprising a first polynucleotide molecule comprising a nucleic acid sequence encoding the CAR and a second polynucleotide molecule comprising a nucleic acid sequence encoding the TEAM.

37. The cell of any one of claims 1-35, comprising a polynucleotide molecule comprising a nucleic acid sequence encoding the CAR and a nucleic acid sequence encoding the TEAM.

38. The cell of claim 37, wherein the polynucleotide molecule further comprises a nucleic acid sequence encoding a linker between the nucleic acid sequence encoding the CAR and the nucleic acid sequence encoding the TEAM, optionally wherein the linker is a cleavable linker.

39. The cell of claim 38, wherein the cleavable linker is self-cleavable, optionally a P2A, E2A, F2A, or T2A self-cleavable linker.

40. The cell of claim 37, wherein the cleavable linker comprises a protease motif.

41. The cell of claim 38, wherein the linker comprises an internal ribosome entry site (IRES).

42. The cell of any one of claims 34-41, wherein the polynucleotide molecule comprises a promoter operably linked to the nucleic acid sequence encoding the CAR and the nucleic acid sequence encoding the TEAM.

43. The cell of any one of claim 42, wherein the promoter is a constitutively active promoter.

44. The cell of any one of claim 43, wherein the promoter is an EF1alpha promoter.

45. The cell of any one of claims 39-44, wherein the sense strand of the polynucleotide molecule comprises, from 5′ to 3′, the nucleic acid sequence encoding the CAR, the linker, and the nucleic acid sequence encoding the TEAM.

46. The cell of any one of claims 39-44, wherein the sense strand of the polynucleotide molecule comprises, from 5′ to 3′, the nucleic acid sequence encoding the TEAM, the linker, and the nucleic acid sequence encoding the CAR.

47. The cell of any one of claims 34-46, wherein the polynucleotide molecule comprises a nucleic acid sequence encoding an amino acid sequence that is at least 85% identical to any one of SEQ ID NOs: 22-25.

48. A polynucleotide comprising a nucleic acid sequence encoding the CAR and the TEAM of any one of claims 37-44.

49. A polypeptide comprising the CAR and the TEAM of any one of claims 1-47.

50. A method comprising administering to a subject the cell of any one of claims 1-47.

51. A method of treating a cancer characterized by cancer cells expressing CD70, the method comprising administering to a subject in need thereof an effective amount of the cell of any one of claims 1-47.

52. A method of treating a cancer characterized by cancer cells expressing CD70 and CD33, the method comprising administering to a subject in need thereof an effective amount of the cell of any one of claims 1-47.

53. A method of treating a cancer characterized by cancer cells expressing CD33, the method comprising administering to a subject in need thereof an effective amount of the cell of any one of claims 1-47.

54. A method of treating a cancer characterized by cancer cells that have decreased CD70 expression, the method comprising administering to a subject in need thereof an effective amount of the cell of any one of claims 1-47.

55. The method of any one of claims 50-54, wherein the subject is human.

56. The method of any one of claims 50-55, wherein administering comprises infusion.

57. The method of any one of claims 51-56, wherein the cancer is a hematological cancer.

58. The method of any one of claims 51-57, wherein the cancer is a myeloid cancer.

59. The method of any one of claims 51-58, wherein the cancer is acute myeloid leukemia.


SEQUENCE LISTING (43 sequences)

SEQ ID NO: 1 - protein, 102 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECD

SEQ ID NO: 2 - protein, 346 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 356 aa, synthetic construct
EQKLISEEDLATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 325 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 344 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDGGGSGGGSGGGSGGGSGGGSGGGSGGGSGGGSGGGSGGGSGGGSGGGSGGGSGGGSGGGSGGGSGGGSILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 296 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 276 aa, synthetic construct
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 9 - protein, 12 aa, synthetic construct
TRPHCESCRHCN

SEQ ID NO: 10 - protein, 20 aa, synthetic construct
ILVIFSGMFLVFTLAGALFL

SEQ ID NO: 11 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 42 aa, synthetic construct
KRGRKKLLYIFKQPFMRPVQTTQEEDGCSCRFPEEEEGGCEL

SEQ ID NO: 13 - protein, 70 aa, synthetic construct
PLPNPSLTARSSQALSPHPQPTHLPYVSEMLEARTAGHMQTLADFRQLPARTLSTHWPPQRSLCSSDFIR

SEQ ID NO: 14 - protein, 19 aa, synthetic construct
MARPHPWWLCVLGTLVGLS

SEQ ID NO: 15 - protein, 21 aa, synthetic construct
MALPVTALLLPLALLLHAARP

SEQ ID NO: 16 - protein, 10 aa, synthetic construct
EQKLISEEDL

SEQ ID NO: 17 - protein, 490 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 18 - protein, 490 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 19 - protein, 111 aa, synthetic construct [sequence omitted]

SEQ ID NO: 20 - protein, 116 aa, synthetic construct [sequence omitted]

SEQ ID NO: 21 - protein, 249 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 22 - protein, 881 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 23 - protein, 881 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 24 - protein, 908 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 25 - protein, 908 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 26 - protein, 12 aa, synthetic construct
GGGSGGGSGGGS

SEQ ID NO: 27 - protein, 15 aa, synthetic construct
GGGGSGGGGSGGGGS

SEQ ID NO: 28 - protein, 20 aa, synthetic construct
GGGGSGGGGSGGGGSGGGGS

SEQ ID NO: 29 - protein, 18 aa, synthetic construct
GSTSGSGKPGSGEGSTKG

SEQ ID NO: 30 - protein, 18 aa, synthetic construct
GGSSRSSSSGGGGSGGGG

SEQ ID NO: 31 - protein, 24 aa, synthetic construct
SGGGGEGRGSLLTCGDVEENPGPR

SEQ ID NO: 32 - protein, 22 aa, synthetic construct
GSGATNFSLLKQAGDVEENPGP

SEQ ID NO: 33 - protein, 21 aa, synthetic construct
METDTLLLWVLLLWVPGSTGD

SEQ ID NO: 34 - protein, 4 aa, synthetic construct
GGGS

SEQ ID NO: 35 - protein, 80 aa, synthetic construct
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 36 - protein, 28 aa, synthetic construct
GGGSGGGSGGGSGGGSGGGSGGGSGGGS

SEQ ID NO: 37 - protein, 8 aa, synthetic construct
GGGSGGGS

SEQ ID NO: 38 - protein, 16 aa, synthetic construct
GGGSGGGSGGGSGGGS

SEQ ID NO: 39 - protein, 20 aa, synthetic construct
GGGSGGGSGGGSGGGSGGGS

SEQ ID NO: 40 - protein, 24 aa, synthetic construct
GGGSGGGSGGGSGGGSGGGSGGGS

SEQ ID NO: 41 - protein, 10 aa, synthetic construct
GGGGSGGGGS

SEQ ID NO: 42 - protein, 25 aa, synthetic construct
GGGGSGGGGSGGGGSGGGGSGGGGS

SEQ ID NO: 43 - protein, 30 aa, synthetic construct
GGGGSGGGGSGGGGSGGGGSGGGGSGGGGS